(19) United States
(12) Patent Application Publication
(10) Pub. No.: US 2025/0312386 A1
(43) Pub. Date: Oct. 9, 2025

(54) Germinal Center Response to Engineered Bacteria Promotes Bladder Cancer Immunotherapy

(71) Applicant: The Trustees of Columbia University in the City of New York, New York, NY (US)

(72) Inventors: Nicholas ARPAIA, New York, NY (US); Tal DANINO, Brooklyn, NY (US); Mathieu ROUANNE, New York, NY (US)

(73) Assignee: The Trustees of Columbia University in the City of New York, New York, NY (US)

(21) Appl. No.: 19/171,839

(22) Filed: Apr. 7, 2025

Related U.S. Application Data
(60) Provisional application No. 63/575,295, filed on Apr. 5, 2024.

(51) Int. Cl. A61K 35/74; A61K 38/19; A61K 39/00; A61P 35/00; C07K 16/28; C12N 1/20; C12R 1/19

(52) U.S. Cl. CPC A61K 35/74; A61K 38/195; A61P 35/00; C07K 16/2818; C07K 16/2827; C12N 1/20; A61K 2039/505; C07K 2317/14; C12R 2001/19

(57) ABSTRACT

E. coli Nissle 1917 (EcN) engineered to express human chemokine CXCL13 (EcN^{hCXCL13}) for intravesical delivery to treat muscle-invasive bladder cancer and similar disorders.

### CROSS-REFERENCE TO RELATED APPLICATIONS

Priority is claimed to U.S. Provisional Application No. 63/575,295 filed 5 Apr. 2024, hereby incorporated by reference in its entirety.

### STATEMENT REGARDING FEDERALLY SPONSORED RESEARCH OR DEVELOPMENT

This invention was made with government support under CA259634 awarded by the National Institutes of Health. The government has certain rights in the invention.

### TECHNICAL FIELD OF THE INVENTION

This disclosure generally relates to the fields of medicine and immunology. More specifically, the disclosure relates to compositions and methods for treating muscle-invasive bladder cancer and related disorders.

### BACKGROUND OF THE INVENTION

Recent evidence suggests that anti-programmed cell death protein 1 (PD-1) immune checkpoint blockade (ICB), a therapeutic monoclonal antibody which promotes durable tumor regression across multiple cancer types, acts not only at the tumor site but also in the draining lymph nodes, where it activates a germinal center (GC) response. The GC reaction is a fundamental component of adaptive immunity, during which antibody affinity and the generation of immunological memory occur in lymphoid organs in response to local antigenic challenge. Although intratumoral microbes have been identified as an intrinsic component of the tumor microenvironment (TME) across human cancer types, their effect on the host humoral immune response during ICB immunotherapy remains poorly understood, which further hinders the ability to develop more effective therapeutic interventions.

Bacillus Calmette-Guérin (BCG) immunotherapy for bladder cancer is the only bacterial cancer therapy approved for clinical use. Upon intravesical delivery, BCG induces an antitumor effect that is dependent on tumor-specific CD4⁺ T cells, similar to most recently employed ICB strategies with regard to the induction of tumor-specific immunity. Emerging evidence has revealed that the presence of intratumoral E. coli in human tumors (including bladder cancer) was associated with improved survival in response to PD-1 blockade in independent clinical cohorts, suggesting a link between intratumoral bacteria and response to immunotherapy. E. coli is the most common bacteria observed in the urinary system and has been found to induce robust immunogenic cellular and humoral immune responses during urinary tract infections. Accordingly, probiotics synthetically engineered to express immunotherapeutic payloads in combination with PD-1 blockade represents a promising strategy to leverage immune activation for the treatment of bladder cancer.

### BRIEF SUMMARY OF THE INVENTION

The present disclosure relates to a probiotic, non-pathogenic strain of E. coli Nissle 1917 (EcN) engineered (or programmed) to express the human chemokine CXCL13 (EcN^{hCXCL13}) for intravesical delivery to treat muscle-invasive bladder cancer and similar disorders. The CXCL13-CXCR5 chemokine axis plays a central role in GC formation by organizing B cell follicles, and recently has been associated with immunotherapy response across multiple tumor types, including bladder cancer. In addition, circulating CXCL13 levels, a plasma biomarker of GC activity, has been found to be positively correlated with early response to PD-1 blockade in bladder cancer patients.

Programmable bacterial cells described herein comprise a (a) a synchronized lysis circuit comprising a first nucleic acid encoding a quorum-sensing gene, a second nucleic acid encoding a lysis gene, a promoter, and a terminator contained on a single operon; and (b) a third nucleic acid encoding CXCL13. In some embodiments, the programmable bacterial cell further comprises a fourth nucleic acid encoding a therapeutic agent selected from the group consisting of an anti-PD-1 antibody and an anti-PD-L1 antibody.

In some embodiments, the programmable bacterial cells belong to at least one genus selected from the group consisting of Salmonella, Escherichia, Firmicutes, Bacteroidetes, Lactobacillus, and Bifidobacteria. In some embodiments, the programmable bacterial cells belong to the genus Escherichia. In particular embodiments, the programmable bacterial cells are Escherichia coli Nissle 1917 (EcN) cells.

The present disclosure also relates to methods of treating muscle-invasive bladder cancer in a subject comprising administering a programmable bacterial cell disclosed herein to the subject. In some embodiments, a plurality of programmable bacterial cells are administered to the subject at least four times. In some embodiments, the plurality of the programmable bacterial cells are administered to the subject once a week for at least four weeks.

In some embodiments, the programmable bacterial cells may be administered to a subject or delivered to the muscle-invasive bladder cancer in the form of a pharmaceutical composition, which may comprise one or more pharmaceutically acceptable carriers, diluents, or excipients.

The present disclosure also relates to articles of manufacture useful for treating muscle-invasive bladder cancer. In some embodiments, the articles of manufacture comprise a container comprising programmable bacterial cells described herein, or pharmaceutical compositions comprising the same, as well as instructional materials for using the same to treat a muscle-invasive bladder cancer. In some embodiments, the articles of manufacture are part of a kit that comprises a bacterial culture vessel and/or bacterial cell growth media.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description of the Invention. This summary is not intended to identify key features or essential features of the claimed subject matter, nor is it intended to be used as an aid in determining the scope of the claimed subject matter.

### BRIEF DESCRIPTION OF THE SEVERAL VIEWS OF THE DRAWINGS

FIGS. 1A-1H show that engineered E. coli Nissle 1917 release human chemokine CXCL13 and promote germinal center reaction in bladder tumor-draining lymph nodes. (A) Schematic demonstrating the release of the human chemokine CXCL13 in bladder tumors after tumor colonization by engineered probiotic E. coli Nissle 1917 upon intravesical delivery (left panel). PBS or MB49 cells (2×10⁵) were implanted in the bladder of C57/BL6 mice. Intravesical delivery of 3×10⁶ CFU of EcN^{hCXCL13} was performed on days 7, 14, and 21. On day 28, supernatant from freshly dissociated tumor-free (n=6) or tumor-bearing (n=5) bladders was plated on LB agar plates containing kanamycin. Colony-forming units (CFU) were counted after overnight culture at 37° C. and computed as CFU per mg of bladder tissue. Bladder tumor-draining lymph nodes were dissociated for immune phenotyping by flow cytometry (right panel). (B) Quantification of the total number of CFU per bladder weight (mg) after overnight culture at 37° C. on LB agar plate with kanamycin. Data shown represent two independent experiments with a total of 5-6 mice per group (**P<0.01, two-tailed unpaired Mann Whitney test). (C) Representative dot plots showing total CD19⁺B220⁺B cells in draining lymph nodes from bladder tumor-free (left panel) and bladder tumor-bearing (right panel) mice. (D) Flow cytometric quantification of total CD19⁺B220⁺B cells among live CD45⁺ cells in draining lymph nodes from bladder-tumor-free and bladder-tumor bearing mice (*P<0.05, two-tailed unpaired Student's t test). (E) Flow cytometric quantification of CD95⁺GL7⁺ germinal center B cells among total B cells in draining lymph nodes from bladder tumor-free and bladder tumor-bearing mice (two-tailed unpaired Student's t test). (F) Representative dot plots showing Bcl6⁺PD-1⁺T follicular helper cells among TCRβ⁺CD4⁺FoxP3⁻ cells in draining lymph nodes from bladder tumor-free (left panel) and bladder tumor-bearing (right panel) mice. (G) Flow cytometric quantification of Bcl6⁺PD-1⁺T follicular helper cells among TCRβ⁺CD4⁺FoxP3⁻ cells in draining lymph nodes from bladder tumor-free and bladder tumor-bearing mice (**P<0.01, two-tailed unpaired Student's t test). (H) Mean fluorescence intensity (MFI) quantification of PD-1 molecules expressed at the surface of Bcl6⁺PD-1⁺T follicular helper cells by flow cytometry in draining lymph nodes from bladder tumor-free and bladder tumor-bearing mice (**P<0.01, two-tailed unpaired Student's t test).

FIGS. 2A-2F show how engineered E. coli Nissle 1917 release a functional human chemokine CXCL13 for bladder cancer therapy. (A) Schematic showing probiotic E. coli Nissle engineered with a synchronized lysis integrated circuit (SLIC) and transformed with a plasmid to produce the human chemokine CXCL13 (EcN^{hCXCL13}) (top). Human CXCL13 is expressed from an Axe/Txe stabilized plasmid under a constitutive tac promoter. The plasmid contains the kanamycin resistance gene (kanR) (bottom). (B) Quantification of the total amount of human CXCL13 by ELISA in culture supernatants of hCXCL13-expressing EcN with (“+”) or without (“−”) SLIC, showing the SLIC-dependent production of hCXCL13. Data represent two independent experiments (**P<0.01, two-sided unpaired Student's t test). (C) Bacteria growth in lysogeny broth (LB) medium subcultured at 1:100 dilution with appropriate antibiotics in a shaking incubator at 150 rpm −37° C., showing no difference of bacteria growth in vitro as compared with the wild-type strain. (D) Flow cytometric quantification of the migration of human B cells in response to bacteria lysates of EcN wild-type, or EcN plus recombinant mouse CXCL13, or EcN^{hCXCL13} (left panel). Flow cytometric quantification of mouse lymphocytes migrating in response to each of the indicated EcN strain lysates in a chemotaxis assay (right panel). Media without any bacteria was used as a negative control. Data shown represent two independent experiments (*P<0.05; **P<0.01; ***P<0.001; ****P<0.0001; two-way ANOVA with Tukey post hoc test). All data are displayed as means±SEM. (E) MB49 cells (2×10⁵) cells were implanted in the bladder of C57/BL6 mice. Intravesical delivery of 3×10⁶ CFU of EcN^{hCXCL13} was performed at day 7, 14, and 21. On day 28, supernatant from freshly dissociated bladder tumor-bearing mice (n=4) was plated on LB agar plates containing kanamycin. Independent colonies were picked up after overnight culture and grown in LB media containing kanamycin (top). The total amount of hCXCL13 after lysis induction for 2 hours at 37° C. in subcultures was quantified by ELISA. EcN⁻ was used as a negative control. Data represent one experiment from n=4 bladders (bottom) (**P<0.01, two-sided unpaired Student's t test). (F) UPPL1541 cells (10 ×10⁶) were implanted in the bladder of wild-type C57BL/6 female mice. Intravesical delivery of either 3 ×10⁶ CFU of EcN^{hCXCL13}, EcN⁻ control (empty vector), or PBS was performed at days 3, 6, 9, and 12. The body weight of mice was monitored as a proxy for mouse health from the start of bacteria treatment and there was no significant weight loss observed in mice treated with EcN^{hCXCL13}, EcN⁻ or PBS. Body weight data represent mean±SEM of n=5-8 biological replicates per group.

FIGS. 3A-3E show that EcN^{hCXCL13} synergizes with PD-1 blockade to promote long-term survival and durable protection in an orthotopic model of advanced bladder cancer. (A) MB49 cells (2×10⁵) were implanted in the bladder of wild-type C57BL/6 female mice. Intravesical delivery of either 3×10⁶ CFU of EcN^{hCXCL13}, EcN⁻ control (empty vector), or PBS was performed at days 3, 6, and 9. Survival was monitored until day 100. (B) Survival curves are shown for each treatment group. Data represent one experiment with n=9-10 mice per group (log-rank test). (C) MB49 cells (2×10⁵) were implanted in the bladder of wild-type C57BL/6 female mice. At day 10, the volume of bladder tumors was monitored by ultrasound imaging. Mean pre-treatment tumor volume was ˜30 mm³. Intravesical delivery of either 3×10⁶ CFU of EcN_{slic}^{hCXCL13}, EcN⁻ control (empty vector), or PBS was performed at day 10, 17, 24, 31. Intraperitoneal injections of PD-1 blockade were done on days 10, 14, 17, and 21. Isotype control IgG was combined with EcN^{hCXCL13} as an additional control. Survival was monitored for >150 days. (D) Survival curves are shown for each treatment group. Tumor clearance was confirmed by ultrasound imaging of the bladder in all surviving mice >150 days. Data represent three independent experiments with n=11-15 mice per group (log-rank test). (E) To assess long-term antitumor control, surviving mice from (D) or age-matched control mice were subcutaneously implanted into both hind flanks with MB49 cells (2×10⁵). Tumor volume was monitored for 41 days post-engraftment (top left panel). Mean tumor growth trajectories are shown in surviving mice previously treated with EcN^{hCXCL13} plus PD-1 blockade and age-matched controls (bottom left panel) (****P<0.0001, two-way ANOVA with Holm-Sidak post hoc test). Individual trajectories of tumor growth are shown in mice from all treatment groups and age-matched controls. MB49 engraftment was observed in mice previously treated with PBS plus PD-1 blockade (n=1/3) and EcN⁻ plus PD-1 blockade (n=1/3). No tumor engraftment was observed in surviving mice previously treated with EcN_{slic}^{hCXCL13} plus PD-1 blockade (n=0/7) (right panel).

FIGS. 4A-4D show that EcN^{hCXCL13} plus PD-1 blockade slow tumor progression and promote CD4⁺T cells and B cells and infiltration in an orthotopic model of PD-1 resistant bladder cancer. (A) UPPL1541 cells (10×10⁶) were implanted in the bladder of wild-type C57BL/6 female mice. Intravesical delivery of either 3×10⁶ CFU of EcN^{hCXCL13}, EcN⁻ control (empty vector), or PBS was performed on day 3, 6, 9 and 12. Bladders were harvested at day 38 for immunofluorescence imaging. (B) UPPL1541 cells (5×10⁵) were implanted in the bladder submucosa of wild-type C57BL/6 female mice using an ultrasound-guided approach. Intravesical delivery of either 3×10⁶ CFU of EcN_{slic}^{hCXCL13}, EcN⁻ control (empty vector), or PBS was performed on day 7, 10, and 13. Intraperitoneal injections of PD-1 blockade were done on day 13, 16, and 19. On day 7 (pre-treatment) and day 21 (post-treatment), the volume of bladder tumors was monitored by ultrasound imaging (data not shown). (C) Day 7 (pre-treatment) and day 21 (post-treatment) tumor volumes of wild-type C57BL/6 female mice are shown. Data represent two independent experiments with n=6 mice per group (****P<0.0001, one-way ANOVA with Holm-Sidak post hoc test). (D) Frequency of CD45⁺TCRβ⁺CD4⁺ cells and CD45⁺TCRβ⁻B220⁺ cell populations from bladder tumors treated as in (C) are shown. Data represent one experiment with mean±SEM of n=3-4 mice per group (**P<0.01, one-way ANOVA with Holm-Sidak post hoc test).

FIGS. 5A-5F show that T follicular helper cells drive the therapeutic efficacy of EcN^{hCXCL13} plus PD-1 blockade. (A) MB49 cells (2×10⁵) were implanted in the bladder of wild-type C57BL/6 female mice. Intravesical delivery of either 3×10⁶ CFU of EcN^{hCXCL13} , EcN⁻ control (empty vector), BCG, or PBS was performed at day 7, 10, and 13. Bladder tumor draining lymph nodes were harvested and dissociated for immune phenotyping by flow cytometry 48 hours after the last intravesical delivery. (B) Quantification of proliferative effector Ki-67⁺CD⁺FoxP3⁻ T helper cells in bladder tumor-draining lymph nodes. Data shown represent two independent experiments with a total of 4-7 mice per group. (****P<0.0001, ***P<0.001, two-way ANOVA with Holm-Sidak post hoc test). (C) Representative flow plots of CD4⁺FoxP3⁻T helper cells co-expressing ICOS⁺PD-1⁺ molecules from bladder tumor draining lymph nodes of mice treated as in (A). (D) Quantfication of activated CD4⁺FoxP3⁻ T helper cells co-expressing ICOS⁺ and PD-⁺ molecules from bladder tumor-draining lymph nodes of mice treated as in (A). Data shown represent two independent experiments with a total of 4-7 mice per group. (*P<0.05, ***P<0.001, ****P<0.0001, two-way ANOVA with Holm-Sidak post hoc test). (E) Eight-week-old CD45.1 mice were irradiated, then reconstituted with bone marrow (BM) from Bcl6^{fl/fl}CD4-cre BL/6 CD45.2 mice. Seven weeks post reconstitution, chimeric mice and age-matched C57/BL6wild-type controls were implanted subcutaneously into each hind flank with MB49 cells (2×10⁵). When tumor volumes reached ˜100 mm3 (day 9), chimeric and control mice were treated with a single intratumoral injection of 1×10⁶ CFU of EcN^{hCXCL13}. Intraperitoneal injections of PD-1 blockade were performed on day 12, 15, and 18. Tumor growth was monitored until day 41. (F) Subcutaneous MB49 tumor growth curves from Bcl6f1/11CD4-cre BL/6 chimeric mice or age-matched C57/BL6 wild-type control mice (n=3-4 per group) that received intratumoral injections of EcN^{hCXCL13} and intraperitoneal PD-1 blockade as in (E). Data represents two independent experiments (****P<0.0001, two-way ANOVA with Holm-Sidak post hoc test).

FIGS. 6A-6F show that EcN^{hCXCL13} plus PD-1 blockade increase central memory T cells in an orthotopic model of advanced bladder cancer. (A) Representative flow plots (left panel) and frequency (right panel) of CXCR5⁺PD-1⁺T follicular helper cells among TCRβ⁺CD4⁺FoxP3⁻ cells from bladder tumor-draining lymph nodes of mice treated as in (B). Bladder tumor-draining lymph nodes were harvested and dissociated for immune phenotyping 48 hours after the last intravesical delivery. (B) MB49 cells (2×10⁵) were implanted in the bladder of wild-type C57BL/6 mice. Intravesical delivery of either 3×10⁶ CFU of EcN^{hCXCL13}, EcN⁻ control (empty vector), or BCG was performed at days 7, 10, and 13. Survival was monitored >day 100. (C) Survival curves are shown for each treatment group. Data represent two independent experiments with n=6-8 mice per group (log-rank test). (D) MB49 cells (2×10⁵) were implanted in the bladder of wild-type C57BL/6 female mice. At day 10, the volume of bladder tumors was monitored by ultrasound imaging. Intravesical delivery of either 3×10⁶ CFU of EcN^{hCXCL13}, EcN⁻ control (empty vector), or PBS was performed at day 10, 17, 24, and 31. Intraperitoneal injections of PD-1 blockade were done on day 10, 14, 17, and 21. Bladder tumor draining lymph nodes were harvested at day 24 for immune phenotyping by flow cytometry. (E) Quantification of Bcl6⁺PD-1⁺ among TCRβ⁺CD4⁺FoxP3⁻ cells in bladder tumor-draining lymph nodes. Data shown represent one experiment with a total of 4-6 mice per group. (*P<0.05, two-way ANOVA with Holm-Sidak post hoc test). (F) Quantification of CD44⁺CD62L⁺ central memory T cell frequency among TCRβ⁺CD4⁺FoxP3⁻ cells (left panel) and number (right panel) in bladder tumor-draining lymph nodes. Data represent one independent experiment with 4-6 mice per group. (*P<0.05, **P<0.01, two-way ANOVA with Holm-Sidak post hoc test).

FIGS. 7A-7G show that EcN^{hCXCL13} plus PD-1 blockade boosts the production of IgG antitumor antibodies. (A) MB49 cells (2×10⁵) were implanted in the bladder of wild-type C57BL/6 female mice. Intravesical delivery of either 3×10⁶ CFU of EcN_{slic}^{hCXCL13}, EcN⁻ control (empty vector), or PBS was performed on day 10 and 17. Intraperitoneal injections of PD-1 blockade were done on day 10, 14, 17, and 21. Bladder tumor draining lymph nodes were harvested and dissociated at day 24 for immune phenotyping by flow cytometry. (B) Quantification of CD95⁺GL7⁺ germinal center B cells among total B cells in bladder tumor-draining lymph nodes. Data shown represent two independent experiments with a total of 3-5 mice per group. (****P<0.0001, two-way ANOVA with Holm-Sidak post hoc test). (C) Representative dot plots showing Bcl6⁺Ki-67⁺ proliferative germinal center B cells among total B cells in draining lymph nodes from bladder-tumor bearing mice. (D) Quantification of Bcl6⁺Ki-67⁺ proliferative germinal center B cells among total B cells in bladder tumor-draining lymph nodes. Data shown represent two independent experiments with a total of 3-5 mice per group. (****P<0.0001, two-way ANOVA with Holm-Sidak post hoc test). (E) Representative dot plots showing activated CD138⁺ plasma cells among CD45⁺ TCRβ⁻ cells. (F) Quantification of the total number of plasma cells in bladder tumor-draining lymph nodes in the treated groups. Data shown represent two independent experiments with a total of 3-5 mice per group. (****P<0.0001, two-way ANOVA with Holm-Sidak post hoc test). (G) MB49 cells were incubated with the serum of mice from each treatment group. The Ig binding affinity of IgG antibodies was quantified by mean fluorescence intensity.

FIGS. 8A and 8B show that EcN^{hCXCL13} plus PD-1 blockade do not induce IgA and IgM antitumor antibodies. (A) MB49 cells (2×10⁵) were implanted in the bladder of wild-type C57BL/6 female mice. Intravesical delivery of either 3×10⁶ CFU of EcN_{slic}^{hCXCL13}, EcN⁻ control (empty vector), or PBS was performed on day 10 and 17. Intraperitoneal injections of PD-1 blockade were done on days 10, 14, 17, and 21. The total level of circulating IgG antibodies was quantified by ELISA. (B) MB49 cells were incubated with the serum of mice from (A) for each treatment group. The Ig binding affinity of IgM and IgA antibodies was quantified by mean fluorescence intensity.

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

In addition, ranges provided in the specification and appended claims include both end points and all points between the end points. Therefore, a range of 1.0 to 2.0 includes 1.0, 2.0, and all points between 1.0 and 2.0.

The term “about” as used herein when referring to a measurable value such as an amount, a temporal duration, and the like, is meant to encompass variations of .+−. 20%, .+−. 10%, .+−. 5%, .+−. 1%, or .+−. 0.1% from the specified value, as such variations are appropriate to perform the disclosed methods.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either”, “one of”, “only one of”, or “exactly one of”.

In the specification and claims, all transitional phrases such as “comprising”, “including”, “having”, “containing”, “involving”, and the like are to be understood to be open-ended, i.e., to mean including but not limited to. Only the transitional phrases “consisting of” and “consisting essentially of” shall be closed or semi-closed transitional phrases, respectively.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

The inventions described herein relate to a strain of E. coli Nissle 1917 (EcN) engineered to express human chemokine CXCL13 (EcN^{hCXCL13}) for intravesical delivery to treat muscle-invasive bladder cancer and similar disorders.

### Programmable Bacteria Cells

In some embodiments of the inventions described herein, cytokines such as human chemokine CXCL13, are produced by one or more programmable bacterial cells. The programmable bacterial cells comprise heterologous nucleic acid sequences, which include one or more sequences that encode the cytokines and sequences that encode a synchronized lysis circuit (i.e., a quorum-sensing gene, a nucleic acid encoding a lysis gene, a promoter, and a terminator contained on a single operon). By including a synchronized lysis circuit, the programmable bacterial cells are capable of lysing in response to one or more internal or external stimuli, such as achieving a certain concentration or cell density in a tumor microenvironment, thereby releasing the synthetic antigens and/or cytokines and other cellular components into the surrounding environment (e.g., tumor microenvironment).

The term “heterologous nucleic acid sequence” refers to a nucleic acid derived from a different organism that encodes for a protein and which has been recombinantly introduced into a cell, In some embodiments, the heterologous nucleic acid sequence is introduced by transformation in order to produce a recombinant bacterial cell. Methods for creating recombinant bacterial cells are well known to those of skill in the art. Such methods include, but are not limited to, different chemical, electrochemical and biological approaches, for example, heat shock transformation, electroporation, liposome-mediated transfection, DEAE-Dextran-mediated transfection, or calcium phosphate transfection. Multiple copies of the heterologous nucleic acid sequence (e.g., between 2 and 10,000 copies) may be introduced into the cell.

In some embodiments, the heterologous nucleic acid sequences are in a plasmid. In some embodiments, the heterologous nucleic acid sequences are in a single operon and are integrated into the genome of the programmable bacterial cells. In some embodiments, the programmable bacterial cells comprise at least one inducible promoter or non-constitutive promoter that is in operable linkage with one or more of the heterologous nucleic acid sequences.

In some embodiments, the programmable bacterial cells comprise one or more biosensor circuits that detect hypoxia, low pH and high lactate levels, which are characteristics of the tumor environment. The biosensor-containing bacterial cells will allow for more specific targeting to the tumor, the biocontainment of the bacterial cells in the tumor and minimize colonization outside the tumor. See, e.g., PCT Application Publication No. WO/2021/137937, hereby incorporated by reference in its entirety.

As used herein, the term “promoter” means at least a first nucleic acid sequence that regulates or mediates transcription of a second nucleic acid sequence. A promoter may comprise nucleic acid sequences near the start site of transcription that are required for proper function of the promoter. As an example, a TATA element for a promoter of polymerase II type. Promoters of the present invention can include distal enhancer or repressor elements that may lie in positions from about 1 to about 500 base pairs, from about 1 to about 1,000 base pairs, from 1 to about 5,000 base pairs, or from about 1 to about 10,000 base pairs or more from the initiation site.

The term “inducible promoter” refers to an operable linkage between a promoter and a nucleic acid sequence, whereby the promoter mediates the nucleic acid transcription in the presence or absence of at least one specific stimulus. In some embodiments, the inducible promoter mediates transcription of a nucleic acid sequence in the presence or absence of at least one, two, three, four, or five or more stimuli. In some embodiments, the one or more stimuli are produced in whole or in part by the programmable bacterial cells. In some embodiments, the only stimulus of the promoter is the presence of a certain concentration or density of programmable bacterial cell found in the subject of a patient (e.g., in a tumor).

An “operable linkage” refers to an operative connection between nucleic acid sequences, such as for example between a control sequence (e.g., a promoter) and another nucleic acid sequence that codes for a protein i.e., a coding sequence. If a promoter can regulate transcription of an exogenous nucleic acid sequence then it is in operable linkage with the gene.

In accordance with the purposes of the inventions described herein, the programmable bacterial cells are preferably non-pathogenic and colonize tumors. One of ordinary skill in the art would know how to attenuate pathogenic bacteria to create non-pathogenic bacteria. In some embodiments, the bacteria are attenuated by removing, knocking out, or mutating a virulence gene such as altering genetic components of the bacterial secretion system.

In some embodiments, the programmable bacterial cells belong to at least one genus selected from the group consisting of Salmonella, Escherichia, Firmicutes, Bacteroidetes, Lactobacillus, and Bifidobacteria. In some embodiments, the bacterial cells belong to more than one genus selected from the group consisting of Salmonella, Escherichia, Firmicutes, Bacteroidetes, Lactobacillus, and Bifidobacteria.

In some embodiments, the programmable bacterial cells belong to the genus Escherichia. In particular embodiments, the programmable bacterial cells are Escherichia col Nissle (EcN) cells.

Some aspects of this invention implicitly relate to culturing the programmable bacterial cells described herein. In some embodiments, a culture comprises the programmable bacterial cells and a medium, for example, a liquid medium, which may also comprise: a carbon source, for example, a carbohydrate source, or an organic acid or salt thereof; a buffer establishing conditions of salinity, osmolarity, and pH, that are amenable to survival and growth; additives such as amino acids, albumin, growth factors, enzyme inhibitors (for example protease inhibitors), fatty acids, lipids, hormones (e.g., dexamethasone and gibberellic acid), trace elements, inorganic compounds (e.g., reducing agents, such as manganese), redox-regulators (e.g., antioxidants), stabilizing agents (e.g., dimethyl sulfoxide), polyethylene glycol, polyvinylpyrrolidone (PVP), gelatin, antibiotics (e.g., Brefeldin A), salts (e.g., NaCl), chelating agents (e.g., EDTA, EGTA), and enzymes (e.g., cellulase, dispase, hyaluronidase, or DNase). In some embodiments, the culture may comprise an agent that induces or inhibits transcription of one or more genes in operable linkage with an inducible promoter, for example doxicycline, tetracycline, tamoxifen, IPTG, hormones, or metal ions. While the specific culture conditions depend upon the particular programmable bacterial cells, general methods and culture conditions for the generation of microbial cultures are well known to those of skill in the art.

### Therapeutic Methods and Compositions

The inventions described herein also encompass methods of treating a muscle-invasive bladder cancer or other related disorder comprising administering to a subject a plurality of programmable bacterial cells described hereinabove. The inventions described herein also encompass methods of reducing the rate of proliferation of a muscle-invasive bladder cancer or other related disorder comprising administering to a subject a plurality of programmable bacterial described hereinabove.

As used interchangeably herein, “treatment” or “treating” or “treat” refers to all processes wherein there may be a slowing, interrupting, arresting, controlling, stopping, alleviating, or ameliorating symptoms or complications, or reversing of the progression of proliferative disease, but does not necessarily indicate a total elimination of all disease or all symptoms. Non-limiting examples of treatment include reducing the rate of growth of a tumor or cancer cell or cell associated with a hyperproliferative disease such as a muscle-invasive bladder cancer or other related disorder, reducing the size of a tumor, or preventing the metastases of a tumor.

Programmable bacterial cells described herein are preferably administered in one or more therapeutically effective doses. As used herein the terms “therapeutically effective dose” means the number of cells per dose administered to a subject in need thereof that is sufficient to treat the hyperproliferative disorder. In some embodiments, a therapeutically effective dose can be at least about 1×10⁴ cells, at least about 1×10⁵ cells, at least about 1×10⁶ cells, at least about 1×107 cells, at least about 1×10⁸ cells, at least about 1×10⁹ cells, or at least about 1x1010 cells.

In some embodiments, programmable bacterial cells may be delivered to a subject in the form of a pharmaceutical composition, which may comprise one or more pharmaceutically acceptable carriers, diluents, or excipients. Pharmaceutical compositions may be formulated as desired using art recognized techniques. Various pharmaceutically acceptable carriers, which include vehicles, adjuvants, and diluents, are readily available from numerous commercial sources. Moreover, an assortment of pharmaceutically acceptable auxiliary substances, such as pH adjusting and buffering agents, tonicity adjusting agents, stabilizers, wetting agents, and the like, are also available. Certain non-limiting exemplary carriers include saline, buffered saline, dextrose, water, glycerol, ethanol, and combinations thereof. Pharmaceutical compositions may be frozen and thawed prior to administration, or may be reconstituted in WFI with or without additional additives (e.g., albumin, dimethyl sulfoxide). Programmable bacterial cells are preferably formulated for parenteral (e.g., intratumoral or intravenous) administration, but other routes of administration known in the art may be utilized.

Particular dosage regimens, i.e., dose, timing, and repetition, will depend on the particular subject being treated and that subject's medical history. Empirical considerations such as pharmacokinetics will contribute to the determination of the dosage. Frequency of administration may be determined and adjusted over the course of therapy, and is based on reducing the number of tumor cells or tumor mass, maintaining the reduction of such tumor cells or tumor mass, reducing the proliferation of tumor cells or an increase in tumor mass, or delaying the development of metastasis.

A therapeutically effective dose may depend on the mass of the subject being treated, his or her physical condition, the extensiveness of the condition to be treated, and the age of the subject being treated.

### Articles of Manufacture

The inventions disclosed herein also encompass articles of manufacture useful for treating a muscle-invasive bladder cancer or other related disorder comprising a container comprising programmable bacterial cells described herein, or a pharmaceutical composition comprising the same, as well as instructional materials for using the same to treat the muscle-invasive bladder cancer or other related disorder. In some embodiments, the articles of manufacture are part of a kit that comprises a bacterial culture vessel and/or bacterial cell growth media.

### EXAMPLES

The following examples have been included to illustrate aspects of the inventions disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

### Mouse Strains

All experiments were performed in compliance with institutional guidelines and were approved by the Columbia University Institutional Animal Care and Use Committee (protocol AC-AABD8554). Seven-week-old wild-type C57BL/6NJ mice were purchased from the Jackson Laboratory, allowed to acclimate for a week, and then injected with cancer cells. Mice were randomized into treatment groups. All mice used were 8 to 12-week-old at time of experiment. Mice were housed at the Herbert Irving Comprehensive Cancer Center or at the Hammer Health Sciences Building under specific pathogen-free conditions.

### Example 2

### Cell Lines

The MB49 mouse bladder cancer cell line was purchased at EMD Millipore Sigma (Catalogue number SCC148). The UPPL1541 mouse bladder cancer cell line was a gift from W. Kim (University of North Carolina at Chapel Hill). The SU-DHL-4 human B cell lymphoma cell line was purchased from The American Type Culture Collection (ATCC CRL-2957). All cell lines tested negative for mycoplasma contamination. The MB49 and UPPL1541 cell lines were cultured inside tissue culture-treated flasks with Dulbecco's modified Eagle medium (DMEM) with 10% fetal calf serum (HyClone) and 1% penicillin-streptomycin (100 U/ml; Gibco), nonessential amino acids, Gluta-MAX, Hepes, sodium pyruvate, and beta-mercaptoethanol. The SU-DHL-4 cell line was cultured in complete RPMI supplemented with 20% FCS and 10% DMSO. Cultures were maintained within a humidified 5% O₂/CO₂ atmosphere at 37° C. inside an incubator. Cells were split twice per week, and cell viability was measured using trypan blue staining.

### Example 3

### Bacteria Generation and Preparation for Intravesical Delivery

The mature bioactive region of the human chemokine CXCL13 (hCXCL13) (Val²³_ Arg⁹⁴, UniProt accession number Q853X90) or the mouse chemokine (mCXCL13) (Ile22-Ala-109, UniProt accession number Q3U1E8) were cloned into plasmid p246 via Gibson Assembly. CXCL13-expressing vectors were transformed into electrocompetent EcN-SLIC strains and cultured in LB media with 50 μg/mL kanamycin with 0.2% glucose, in a 37° C. shaking incubator. For therapeutic preparation, EcN^{hCXCL13} and EcN⁻ (empty vector) strains were grown overnight in LB media containing appropriate antibiotics and 0.2% glucose. The overnight culture was sub-cultured at a 1:100 dilution in 50 mL of fresh media with antibiotics and glucose and grown to an OD600 of ˜0.05, preventing bacteria from reaching quorum. Cells were centrifuged at 3000 rcf and washed 3 times with sterile ice-cold PBS. EcN^{hCXCL13} and EcN⁻ strains were then diluted to a final concentration of 3×10⁷ CFU/mL in cold PBS, and 100 μL of each strain (3×10⁶ CFU) was then injected intravesically. Bacillus Calmette-Guerin (BCG, TICE strain, Merck) was obtained by resuspension in PBS of the lyophilized content of clinically available MSD vials. BCG was delivered into the bladder in a 100 μL volume of phosphate-buffered saline (PBS) containing 3×10⁶ CFU of bacteria.

### Example 4

### Intravesical Tumor Implantation

MB49 and UPPL1541 cells were detached from tissue culture plastic using 0.05% trypsin-EDTA (Gibco) for 1 to 3 min at 37° C. Cells were then washed using PBS, and cell viability and counting was assessed using trypan blue staining. Cells were resuspended in PBS at 2×10⁶ cells/mL for MB49 or 15×10⁶ cells/mL for UPPL1541. Eight-to 12-week-old female mice (Jackson Laboratory) were placed under anesthesia in an isoflurane chamber. A 24-gauge catheter (Terumo) was inserted into the bladder through the urethra. Next, 100 μL of poly-L-lysine (Sigma) was injected in the bladder through the catheter, the catheter was capped using an injection plug (Terumo), and the mice were kept under anesthesia for 30 min. The mouse was removed from the isoflurane chamber, the bladder was manually emptied, and the catheter was removed. The catheter was then flushed with a solution containing 2×10⁵ MB49 cells/mL in PBS or 15×10⁶ cells/mL for UPPL1541 in PBS. Next the catheter was reinserted and 100 μL of the MB49 solution (˜250,000 cells per mouse) or UPPL1541 solution (˜1,500,000 cells per mouse) was injected into the bladder. The mice were kept under anesthesia for 2 hours and were allowed to recover from anesthesia. Mice were then monitored regularly after implantation for signs of hematuria and tumor growth. To generate models of advanced bladder cancer, (I) MB49 cells were grafted 10 days before starting treatment; (II) UPPL1541 cells were implanted in the lamina propria by ultrasound-guided injection at 5×10⁶ cells suspended in 500 μL sterile PBS using 30G needle with syringe as previously described. Ultrasound imaging of the bladder was used to measure the tumor volume pre-and post-treatment. Groups were assigned with matching pre-treatment tumor volume between experimental conditions. Mice were weighed two times weekly and euthanized when the humane endpoint of 20% weight loss was reached, or if they displayed signs of distress, such as dull fur or apathy.

Mice were anesthetized, and 4-gauge plastic catheters were used to instill bacteria into the bladder of mice. A total volume of 100 μL of 3×10⁶ CFU of EcN^{hCXCL13}, EcN⁻, or BCG was injected into the bladder. PBS was used as a control. The catheter was capped using an injection plug (Terumo). The mice were kept under anesthesia for 2 hours. At the end of this time, catheters were removed, and the mice were allowed to recover from anesthesia. Dosing schemes are indicated in each figure and figure legends.

### Example 5

### Subcutaneous Tumor Implantation

To assess immune memory, surviving mice were subcutaneously rechallenged with MB49 cells after MB49 orthotopic implantation. All surviving mice were free of bladder tumors. Mice were injected on both flanks with 100 μL at 2×10⁶ cells/mL of MB49 cells (˜500,000 cells per mouse). Caliper measurements were used to track tumor volume every 3-4 days. Tumor volume was calculated by measuring the length, width and height of each tumor using calipers, where V=(length×width×height×π/6). Mice were euthanized when tumor volumes reached 1,000 mm³.

For intratumoral bacterial injection, a single dose of EcN^{hCXCL13} strain (1×10⁶ CFU) in 40 μL of PBS was injected. Bacteria were injected on day 9 in subcutaneously implanted MB49tumors in both hind flanks.

### Example 6

### Generation of Bone Marrow Chimeric Mice

To deplete T follicular helper cells (Tfh) in mice, full bone marrow (BM) chimeras were generated by reconstituting 8-week-old male C57BL/6 mice with BM from Bcl6^{fl/fl}CD4-cre male C57BL/6 mice provided by Shane Crotty's lab (Center for Infectious Disease and Vaccine Research, La Jolla Institute for Immunology, La Jolla, CA). To achieve complete myeloablation, 8-week-old wild-type C57BL/6NJ male mice were exposed to whole body irradiation (2 doses of 5.25 Gy per mouse, 4 hours apart). After at least 4 h from the last irradiation, mice were reconstituted with 5×10⁶ whole BM cells isolated from donors and injected intravenously by retro-orbital injection. Mice were treated with antibiotics in the drinking water for 4 weeks after reconstitution. Mice were placed in standard cages and allowed to reconstitute for 4 more weeks total before subcutaneous tumor implantation.

### Example 7

### Ultrasound Imaging

Mice were anesthetized using inhalation isoflurane, and hair was removed from their lower abdomen by using chemical hair removal cream. Ultrasound gel was applied to the abdomen, and mouse bladders were imaged using the VEVO 3100 Ultrasound Imaging System (FUJIFILM VisualSonics, Toronto, Canada) located within the mouse barrier in the Herbert Irving Cancer Center Small Animal Imaging facility. Tumor volume was calculated via 3D reconstruction program (Vevo LAB).

### Example 8

### Chemotaxis Assay

To characterize the chemotaxis effect of hCXCL13-derived bacteria on mouse lymphocytes, splenocytes were isolated from wild-type adult C57BL/6 mouse spleen using mechanical dissociation in wash buffer (RPMI 1640 supplemented with 10% FBS, HEPES, Glutamax, Pen/Strep). Cells were filtered through 100 μm cell strainers and resuspended in wash media at a concentration of 1×10⁶ cells per mL. Overnight cultures of each bacterial strain (without SLIC) were grown in LB with appropriate antibiotics and then subcultured at a 1:100 dilution in a shaking incubator for 60 min at 37° C. Bacteria were washed twice in serum-free complete RPMI, matched at optical density at 600 nm (OD₆₀₀), and lysed via sonication in serum-free complete RPMI. Lysates were centrifuged to remove debris (20817 g for 10 min at 4° C.), and 235 μl of the supernatant was entered into the lower chamber of a Corning HTS transwell plate (well area=0.143 cm²; pore size=5 μm). Mouse lymphocytes (75 μl of the preparation described above) were added to the upper chamber, and the plate was incubated for 3 hours in a humidified 37° C. 5% CO₂ incubator. The bottom chamber was then harvested and washed in complete media. Then, samples were acquired on a BD Fortessa for 60 seconds. Cell counts were normalized to the number of cells entered into the assay. To characterize the chemotaxis effect of hCXCL13-derived bacteria on human B cells, SU-DHL4 cells were harvested from cell culture and used as mentioned above.

### Example 9

### Enzyme-Linked Immunosorbent Assay

For in vitro quantification of human CXCL13, overnight cultures of EcN⁻ and EcN^{hCXCL13} were grown in LB agar with or without kanamycin, and then subcultured (1:100 dilution) for 60 minutes in a shaking incubator at 37° C. Bacteria were harvested, concentration matched at optical density at 600 nm (OD₆₀₀), and resuspended in 2 mL LB, followed by continued culturing in a shaking incubator for 60 min at 37° C. in presence of AHL for SLC-induced lysis (20). Cultures were then centrifuged (3000× g for 10 min at 4° C.), and supernatants were entered into a human CXCL13 enzyme-linked immunosorbent assay (ELISA) (R&D SYSTEMS™ Human CXCL13/BLC/BCA-1 DuoSet ELISA, catalog number DY801), performed as per the manufacturer's protocol. To quantify mouse IgG after combined therapy with PD-1 blockade and intravesical EcN^{hCXCL13}, EcN⁻, or PBS, the serum levels of total circulating IgG antibodies were detected and quantified using the Mouse IgG (Total) uncoated ELISA kit (Catalog number 88-50400-22), performed as per the manufacturer's protocol.

### Example 10

### Immunophenotyping by Flow Cytometry

Bladders were dissected, cut into small pieces, and digested in wash media (RPMI 1640 supplemented with 5% FCS, HEPES, Glutamax, Pen/Strep) with 1 mg/mL collagenase A and 0.5 μg/mL DNAse I in a shaking incubator at 37° C. for 45 minutes. Digestion was stopped by adding FACS buffer (PBS supplemented with 1% bovine serum albumin, 2.5 mM EDTA, and 0.1% NaN₃). Single-cell suspensions were washed and resuspended in ice cold FACS buffer prior to staining.

Lymph nodes were homogenized with a syringe plunger and filtered through a 70 mm cell strainer on ice. Cells from all tissues were resuspended in ice cold FACS buffer and immediately used for counting or staining. Live/dead staining was performed via Ghost Dye Red 780 labeling (Tonbo Biosciences), as per the manufacturer's protocol. All staining steps were carried out at 4° C. in FACS buffer (PBS supplemented with 1% bovine serum albumin, 2.5 mM EDTA, and 0.1% NaN₃).

Single cell suspensions were Fc blocked with anti-CD16/CD32 monoclonal antibody prior to staining. Cells were then stained for flow cytometry, with intracellular staining performed using the Tonbo Foxp3 Transcription Factor Staining Buffer Kit as per the manufacturer's protocol. Antibodies used included anti-CD45 (clone 30-F11, BD Biosciences), TCRβ (clone H57-597, BD Biosciences), B220 (clone RA3-6B2, BD Biosciences), CD19 (clone 1D3, Tonbo), GL7 (clone GL7, BD Biosciences), CD95 (clone JO2, BD Biosciences), CD138 (Clone: 281 2, BD Biosciences), CD4 (clone RM4-5, BD Biosciences), CD8 (clone 53-6.7, BioLegend), Foxp3 (clone FJK-16s, eBioscience), CD44 (clone IM7, BioLegend), CD62L (clone MEL-14, Tonbo), Bcl6 (clone K112 91, BD Biosciences), PD-1 (clone 29F.1A12, BioLegend), CXCR5 (clone L138D7, BioLegend), Ki-67 (SolA15, ThermoFischer), IgG (clone Poly4053, BioLegend), IgM (clone R6 60.2, BD Biosciences), IgA (clone C10-3, BD Biosciences). Total cell counts were determined by the addition of counting beads (Invitrogen) to a known volume of sample after staining, just before cytometer acquisition. All samples were acquired on a BD LSR-II or LSR-Fortessa using DIVA software, and data were analyzed in FlowJo (Tree Star) software.

### Example 11

### Immunostaining

Bladders were fixed overnight in 4% paraformaldehyde at 4° C. and embedded in paraffin. Serial sections of 4 mm were generated and mounted on adhesive microscope slides. For immunofluorescence staining, paraffin sections were deparaffinized using HISTOCLEAR® and rehydrated through a series of Ethanol and 1× PBS washes. Antigen retrieval was performed by boiling slides for 15 min in pH 9 buffer or 30 min in pH 6 buffer. Primary antibodies in 1% heat inactivated horse serum and 1% bovine serum albumin were incubated overnight at 4° C. The next day, slides were washed with PBS-Triton 0.5% three times for 10 min each and secondary antibodies were applied for 90 minutes at room temperature. DAPI (4′6-diamidino-2-phenylindole) was applied as part of the secondary antibody cocktail for nuclear staining. Slides were sealed with coverslips using DAKO mounting gel and stored at 4° C. until analysis.

### Example 12

### Quantification of Bacterial Viable Cell Numbers

Bladders were weighed and homogenized in 1 mL of sterile PBS at 4° C. using a handheld rotor-stator tissue homogenizer (TissueRuptor II, Qiagen). Homogenates were serially diluted, plated on LB agar plates and incubated overnight at 37° C. For plasmid retention analysis, tumor homogenates were plated on LB-agar plates containing kanamycin. Colonies were counted and computed as CFU/mg of tissue.

### Example 13

### Detection of Antitumor Antibodies

Whole blood was collected via terminal cardiac puncture, incubated at room temperature for 45 min, and centrifuged at 14000× g (maximum speed) for 15 minutes, 4° C. Serum was recovered and stored at −20° C. for antibody binding assays. To detect antitumor antibodies after therapy, MB49 cells were incubated with sera diluted 1:50 in PBS for 30 min at room temperature, washed with FACS buffer, stained with fluorescently labelled antibodies to mouse IgG, IgA and IgM for 30 min at room temperature and analyzed by flow cytometry on a LSR-II flow cytometer. Antibody titers are represented as the MFI per antibody isotype. Prior to incubation, MB49 cells were washed three times using centrifugation and PBS and resuspended in 100 AL PBS buffer. Sera were normalized at 40 ng/ml IgG in PBS. A pre-absorption protocol was followed to remove autoantibodies as previously described. Briefly, the pre-absorption protocol was done by incubating pre-diluted sera with cells from freshly dissociated normal bladder urothelium from C57BL/6NJ wild-type mice for 45 min on ice. Cells were then centrifuged, the supernatant collected and then incubated with MB49 cells for 45 min on ice. Background was assessed using the antibody against MB49 cells incubated with PBS-no serum.

### Example 14

### Statistical Analysis

Statistical analysis was performed in GraphPad Prism 9, using Student's T test or one- or two-way analysis of variance (ANOVA) with post hoc testing as indicated. A P value of <0.05was considered statistically significant. The specific statistical test, sample sizes, and P values for each figure are indicated in the figure legends.

### Example 15

Programmed E. coli Nissle 1917 Releases Human Chemokine CXCL13 and Promotes the GC Reaction in Bladder Tumor-Draining Lymph Nodes

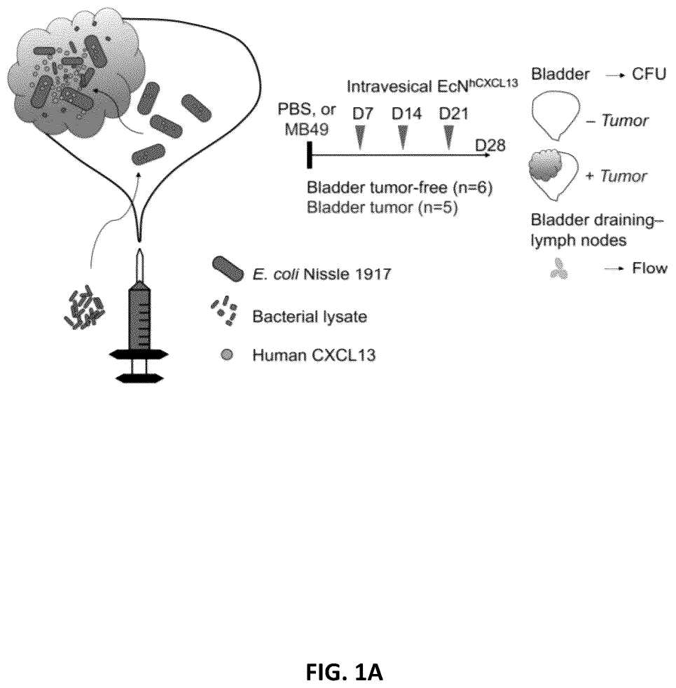

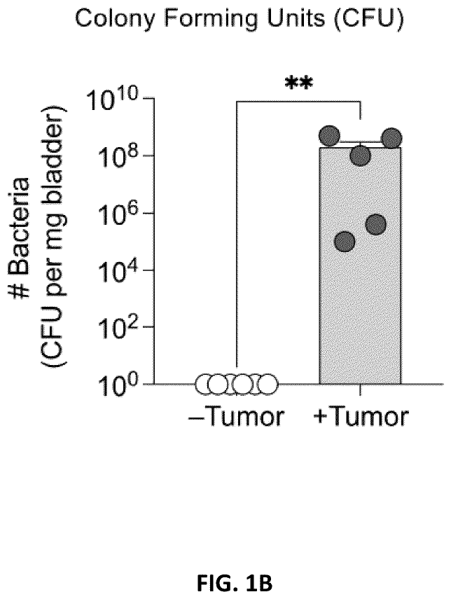

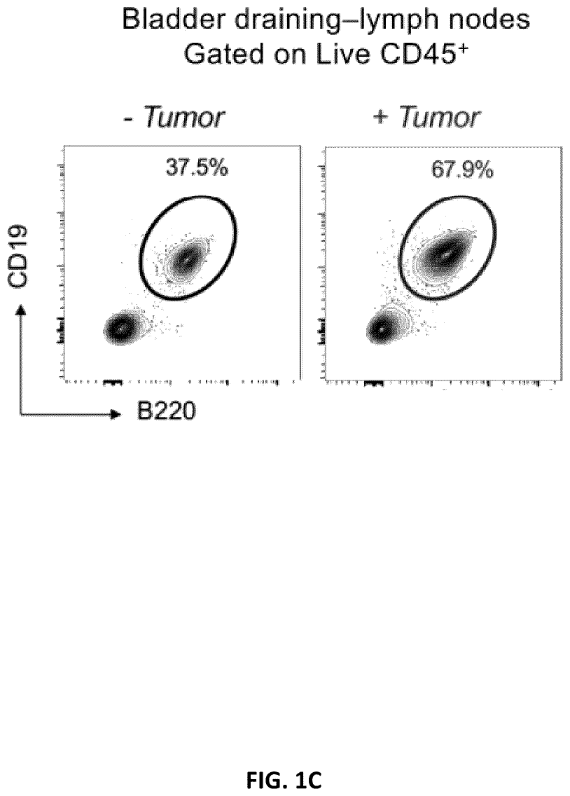

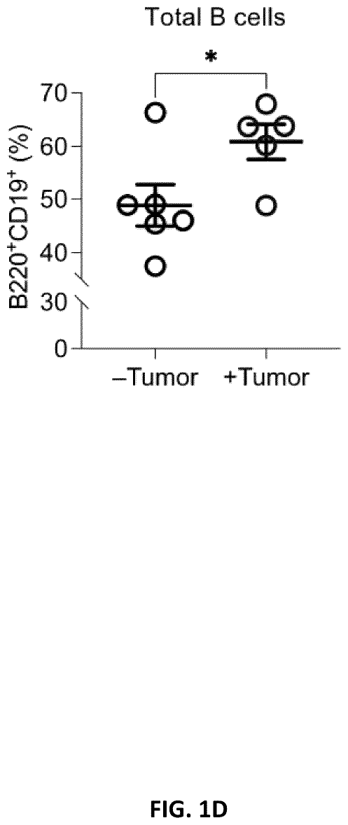

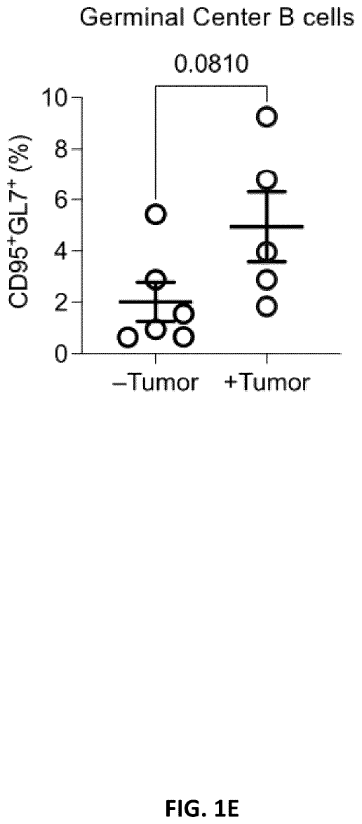

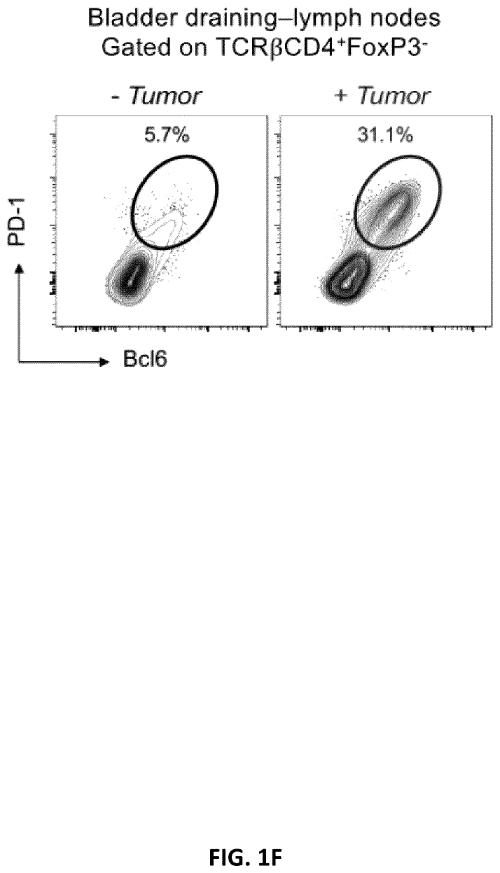

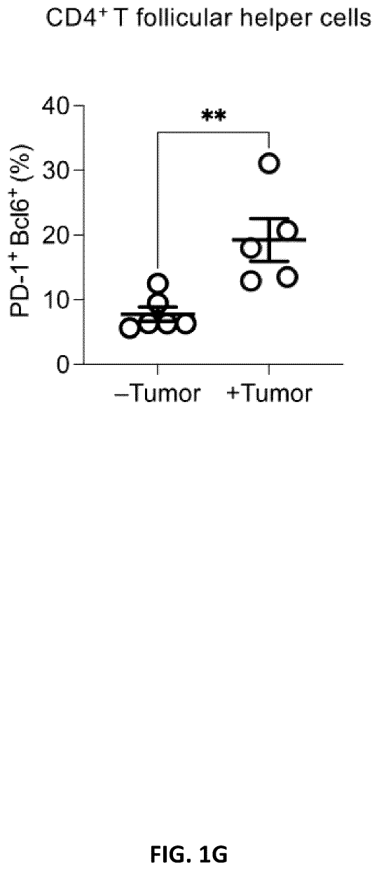

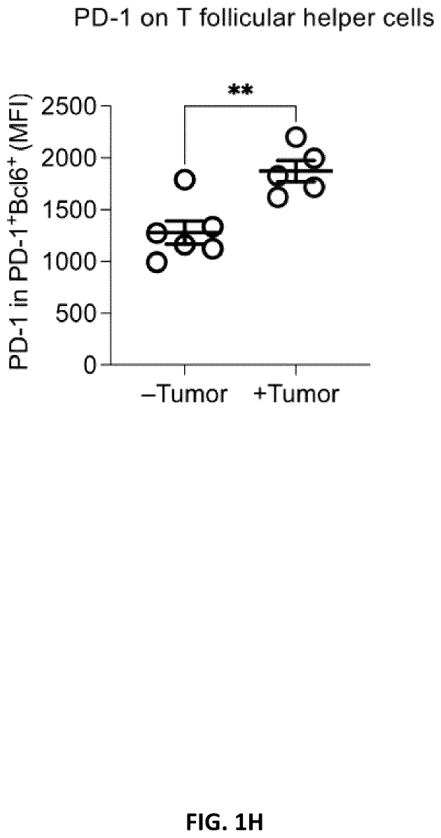

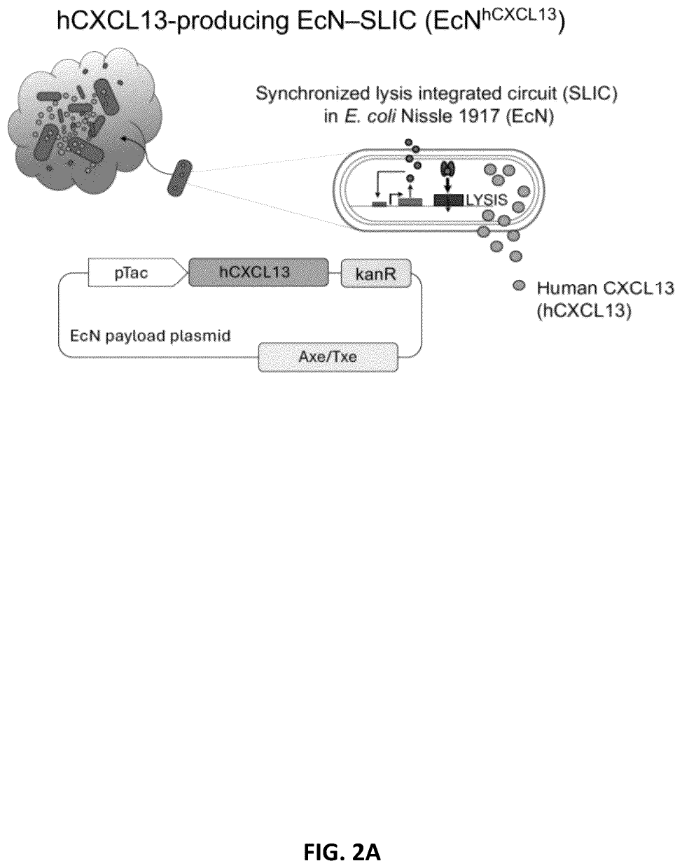

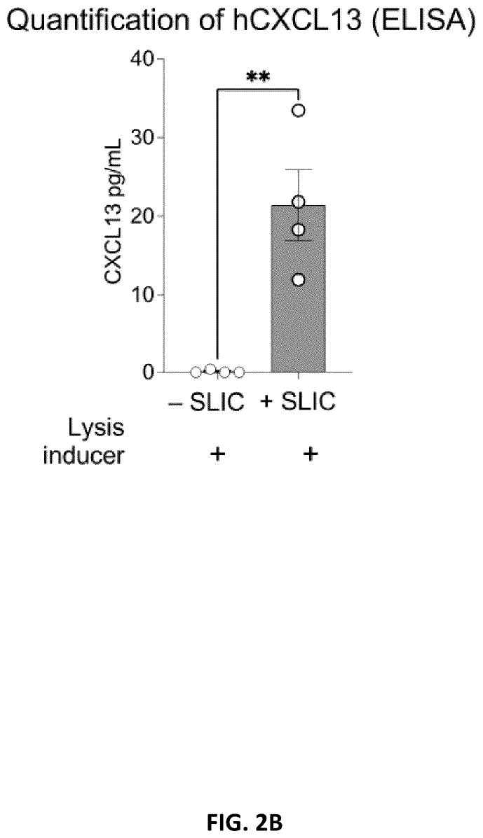

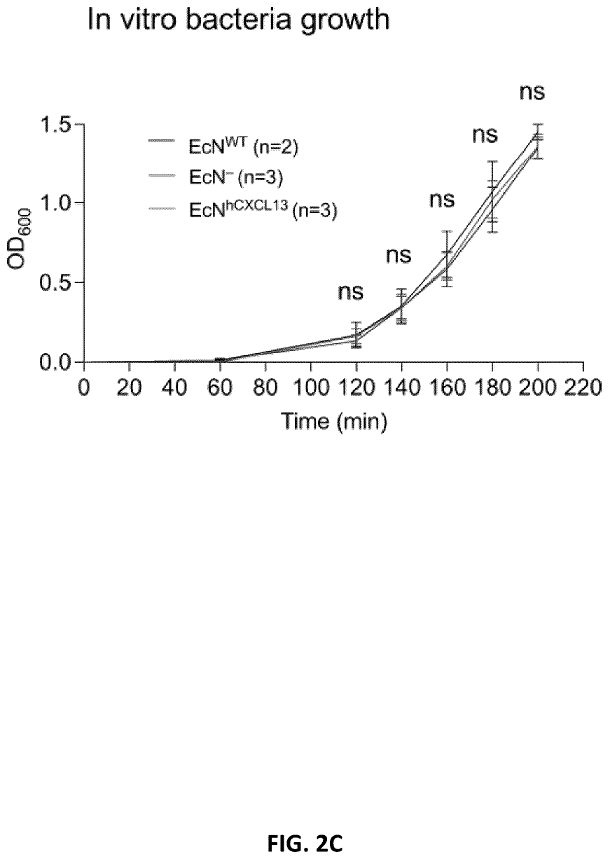

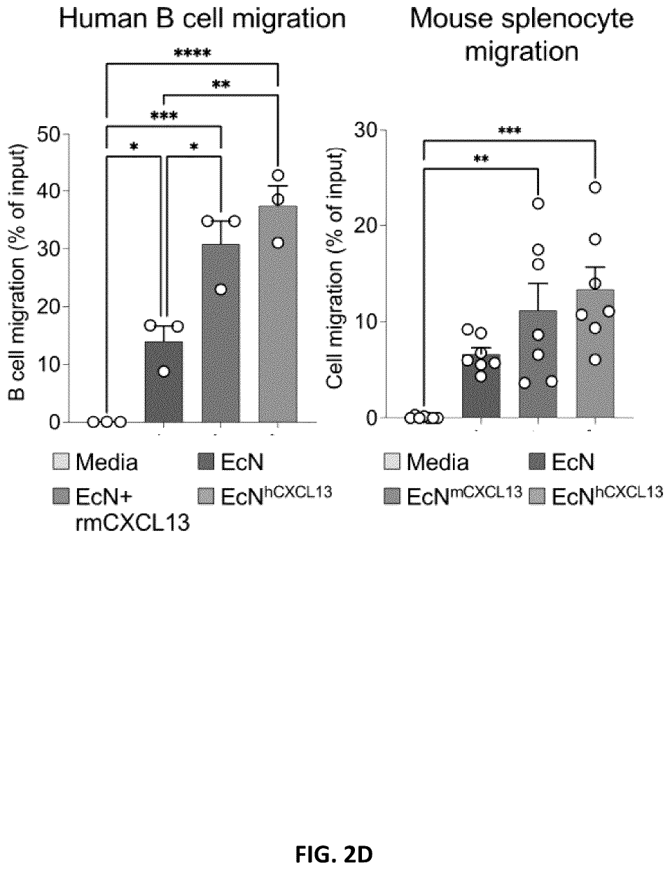

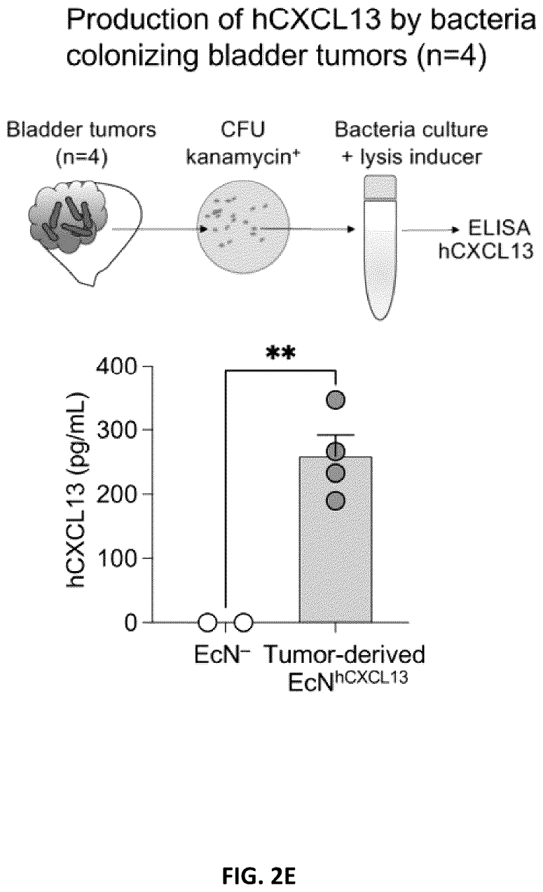

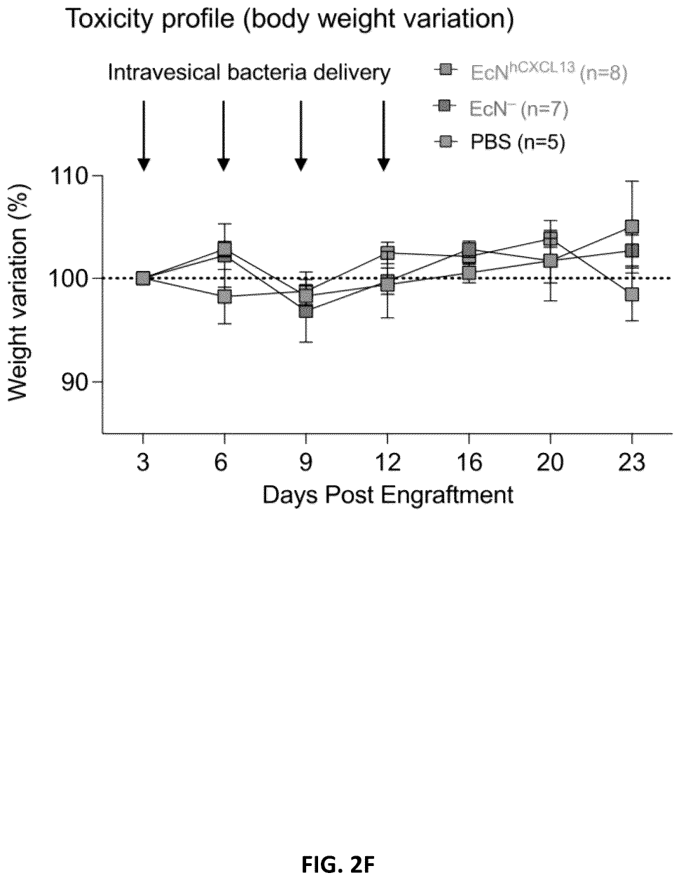

To effectively deliver the human chemokine CXCL13 (hCXCL13) to the bladder TME, Escherichia coli Nissle 1917 (EcN) was engineered with a previously-described synchronized lysis integrated circuit (SLIC) for quorum sensing-triggered release of plasmid-encoded hCXCL13, EcN^{hCXCL13} (FIG. 1A and FIG. 2A). Specifically, bacteria harboring this integrated circuit grow until they reach a critical population density within the TME, therefore triggering cyclic lysis events that release co-encoded payloads, such as chemokines, in situ. Release of hCXCL13 was SLIC dependent and EcN^{hCXCL13} demonstrated no difference in growth rate relative to EcN^{WT} in non-lysing conditions (FIG. 2C and 2B). Taken together, these data suggest that incorporation of both a hCXCL13-expressing plasmid and the integrated SLIC system did not affect bacterial growth as compared to the wild-type EcN.

To further evaluate the functionality of hCXCL 13 produced by the engineered bacteria, a chemotaxis assay was performed in which the migration of human B cells expressing high levels of the chemokine receptor CXCR5 was evaluated in response to lysates of EcN^{hCXCL13}, EcN expressing mouse CXCL13 (EcN^{mCXCL13}), or control EcN (23). Compared to control EcN lysate, human B cells significantly migrated in response to lysates of both EcN^{hCXCL13} and EcN^{mCXCL13} (FIG. 2D). Similarly, mouse splenocytes migrated to a greater extent to lysate from EcN^{hCXCL13} (FIG. 2D). Thus, we used EcN^{hCXCL13} to facilitate future translation to human studies.

To generate bladder tumors, we intravesically implanted MB49 cells in syngeneic wild-type mice, a well-studied orthotopic mouse model of bladder cancer with an aggressive phenotype. To assess the ability of EcN^{hCXCL13} to effectively and specifically colonize bladder tumors upon intravesical delivery, bladder tumor-bearing and tumor-free mice were exposed to repeated intravesical instillations of engineered bacteria once a week for 3 weeks (FIG. 1A) Notably, colonies of EcN^{hCXCL13} were observed only in bladder tumors and were not found in tumor-free bladders after intravesical delivery (FIG. 1B). Furthermore, bacterial colonies isolated from freshly dissociated bladder tumors were able to grow under selective antibiotic pressure and release the human chemokine CXCL13, confirming their ability to maintain therapeutic plasmids while growing in vivo (FIG. 2E). These observations demonstrate that bacteria preferentially colonize bladder tumors while sparing non-tumoral tissue and consistent with prior preclinical data. Furthermore, no significant weight loss was observed in mice treated with EcN^{hCXCL13}, EcN⁻ control (empty vector), or PBS (FIG. 2F), confirming the favorable safety profile of our therapeutic approach.

To characterize the immune response to this strain of EcN, immunophenotyping of T cell and B cell infiltrates was performed in the draining lymph nodes from bladder tumor-bearing and tumor-free mice after intravesical delivery of EcN^{hCXCL13}, with the aim of assessing the GC response. An increased frequency of total B cells and GC B cells, as well as an increase in Tfh cells, was detected in bladder tumor-draining lymph nodes as compared with bladder tumor-free mice (FIG. 1C-1G). A significant increase of cell surface PD-1 molecules on Tfh cells in bladder tumor draining lymph nodes was observed. These data suggest that intravesical delivery of EcN^{hCXCL13} actively promotes the GC reaction in draining lymph nodes from bladder tumor-bearing mice, most likely due to the in situ release of hCXCL13 in bladder tumors colonized by EcN^{hCXCL13} (FIG. 1H). Together, these observations demonstrate both the safety and ability for intravesically-delivered EcN^{hCXCL13} to elicit the GC reaction in orthotopic models of bladder cancer.

### Example 16

EcN^{hCXCL13} Synergizes with PD-1 Blockade to Promote Long-Term Survival and Durable Protection in an Orthotopic Model of Advanced Bladder Cancer.

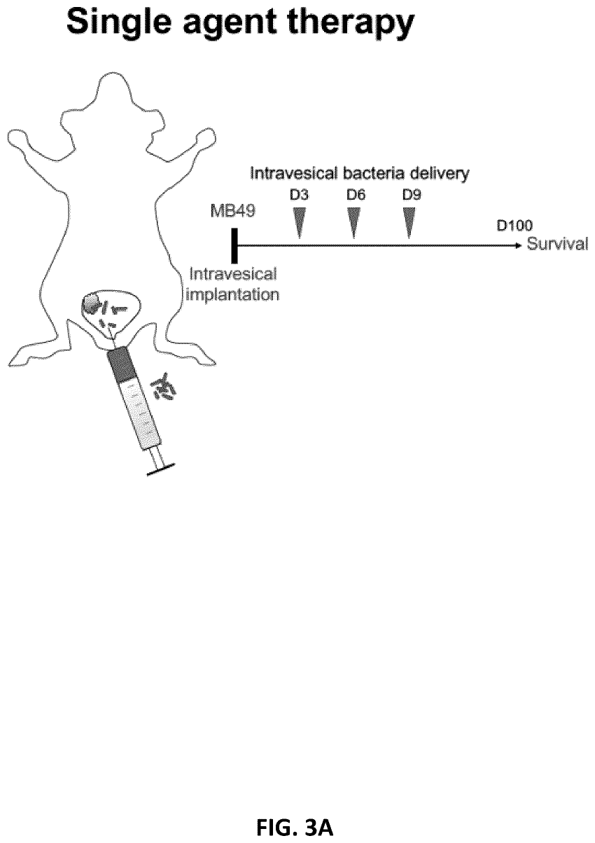

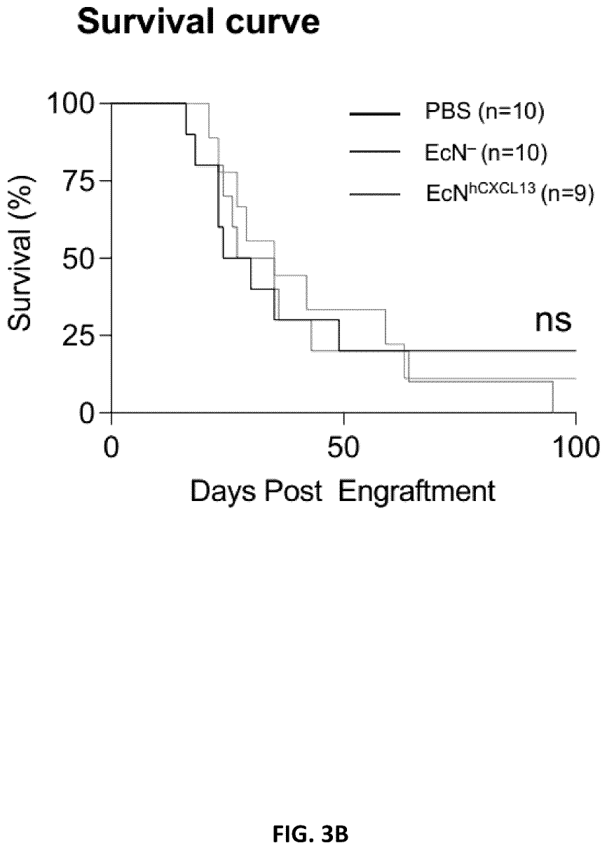

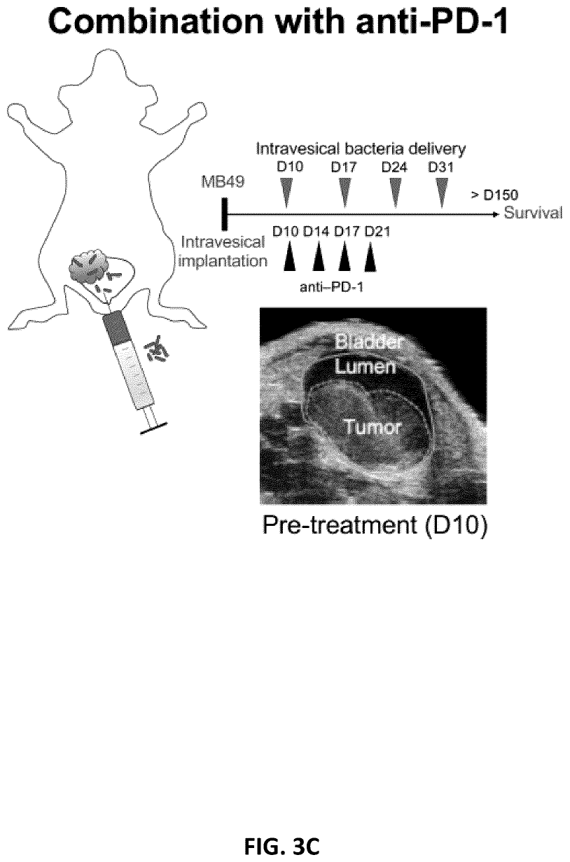

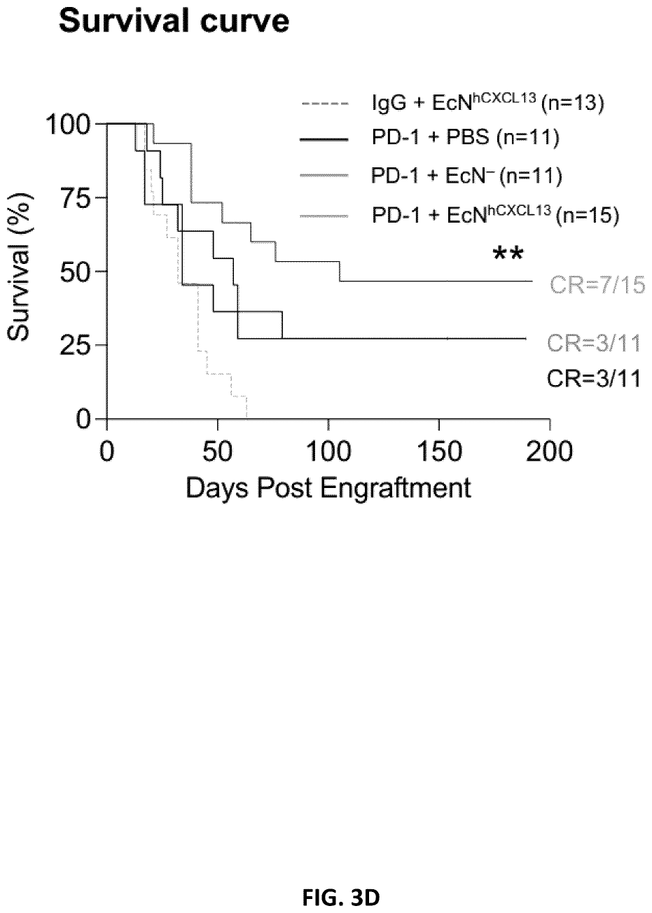

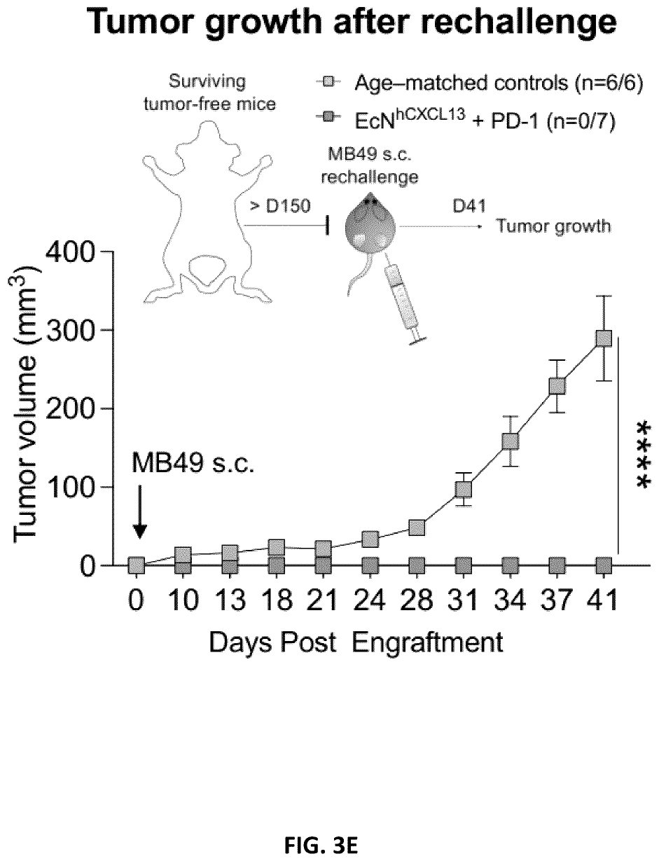

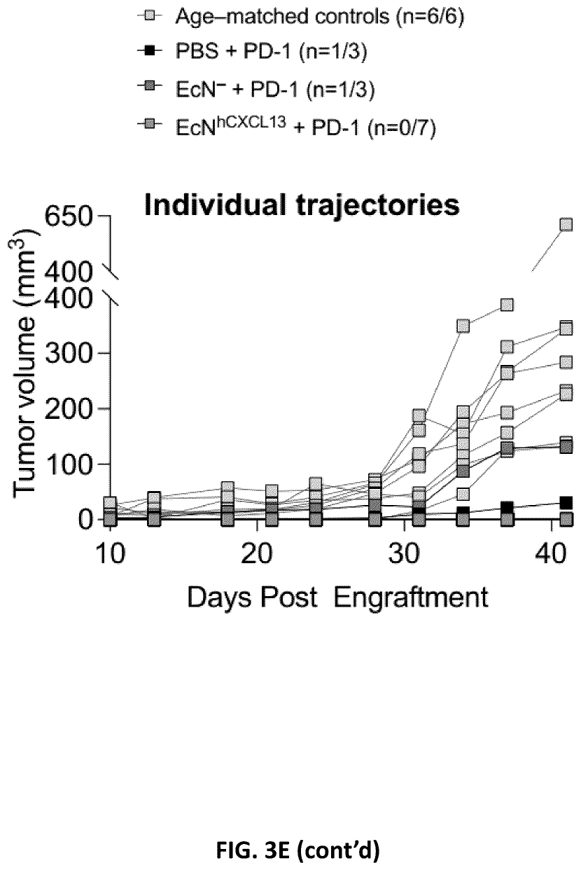

EcN^{hCXCL13} can sensitize tumors to PD-1 blockade by activating the GC reaction in bladder tumor-draining lymph nodes in response to PD-1 binding on Tfh cells. In the absence of PD-1 blockade, the median survival of MB49 bladder tumor-bearing mice treated with intravesical delivery of EcN^{hCXCL13} at days 3, 6, and 9 post-tumor implantation was 35 days, compared with 31 and 27 days for mice treated with EcN⁻ or PBS, respectively (FIG. 3A and 3B). Significant antitumor activity of EcN^{hCXCL13} was not observed as a single agent in the MB49 orthotopic model. However, in a separate established orthotopic model of bladder cancer using the UPPL1541 cell line, a spatial reorganization of immune cells in the bladder mucosa was consistently noted as compared with animals treated with control EcN or PB. Induction of immune aggregates of CD4⁺ T cells, CD138⁺ plasma cells and F4/80⁺ macrophages 4 weeks after repeated intravesical instillations of EcN^{hCXCL13} was observed. These data suggest that EcN^{hCXCL13} was biologically and immunologically active at the mucosal surface of the bladder upon intravesical delivery but was not sufficient as a single therapeutic agent to induce a significant survival benefit.

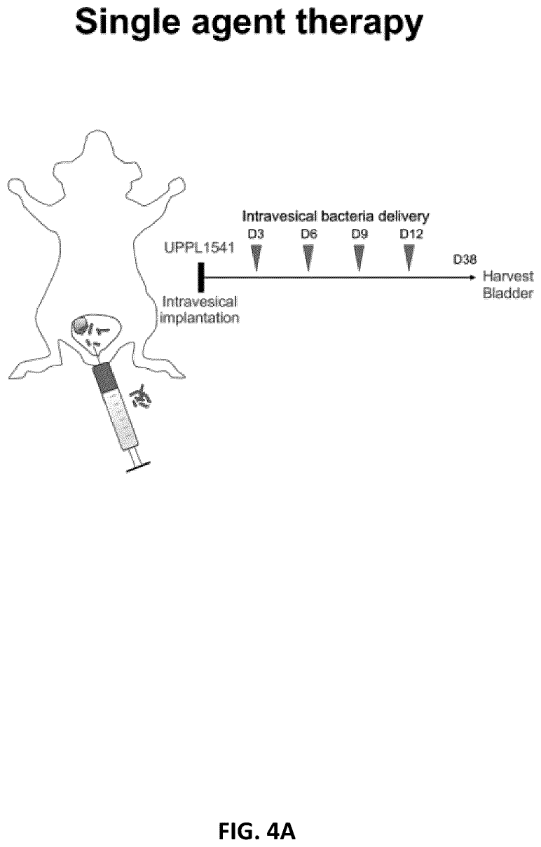

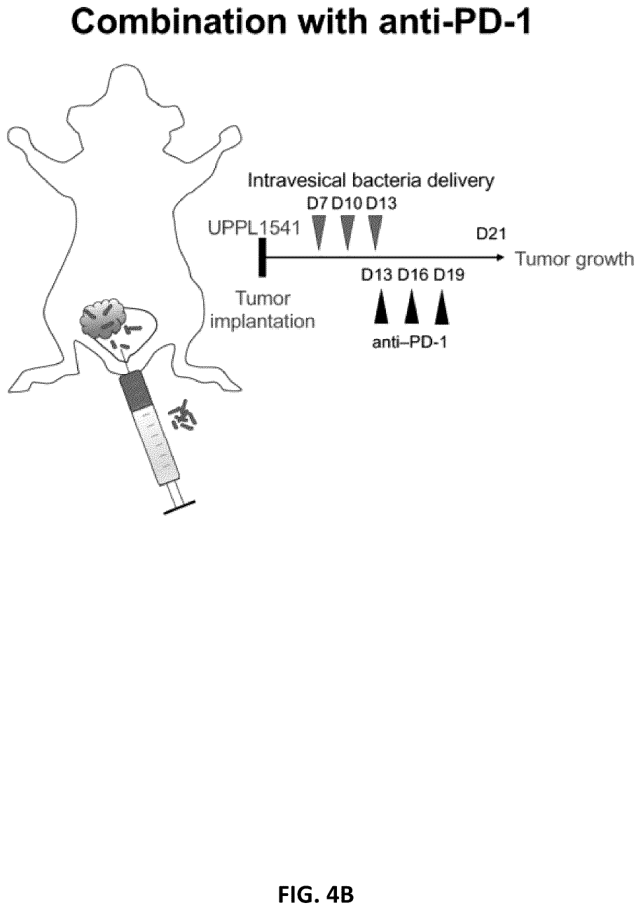

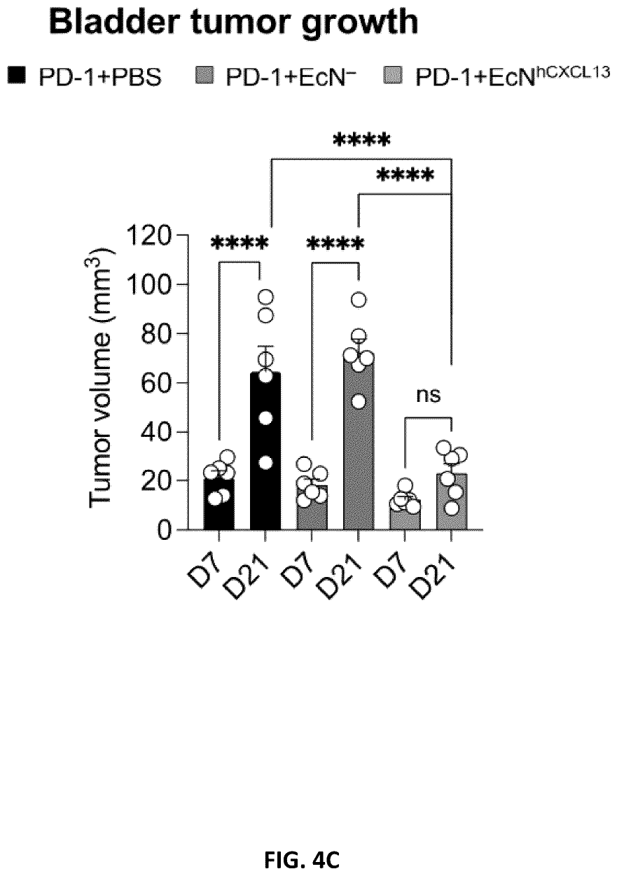

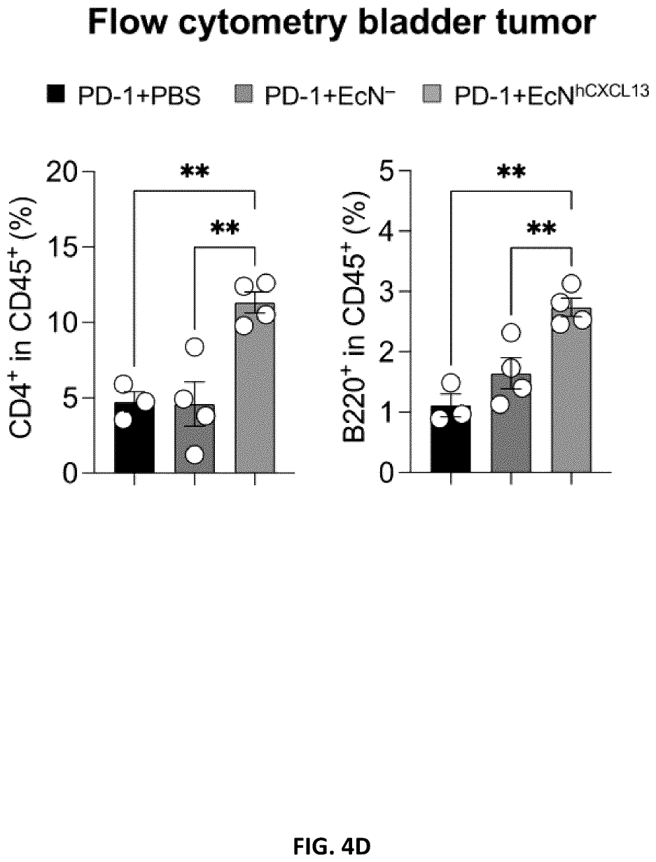

To further assess the antitumor efficacy of EcN^{hCXCL13} combined with PD-1 blockade in an orthotopic model of advanced bladder cancer with previously described primary resistance to PD-1 blockade, UPPL1541 cells were implanted in the bladder submucosa of mice using an ultrasound-guided approach (FIG. 4A). In this setting, mice were intravesically treated EcN^{hCXCL13}, EcN⁻, or PBS at days 7, 10, and 13, along with repeated injections of PD-1 blockade at days 13, 16, and 19. The tumor volumes between pre-treatment day 7 and post-treatment day 21 was evaluated by ultrasound imaging of the bladder to assess the dynamics of bladder tumor growth (data not shown). Notably, EcN^{hCXCL13} plus PD-1 blockade significantly restrained tumor growth when compared with UPPL1541 bladder tumors treated with EcN⁻ control or PBS plus PD-1 blockade (FIG. 4B). Furthermore, a significant increase in the frequencies of total B cells and CD4+ T cells was observed infiltrating the bladder tumors upon EcN^{hCXCL13} plus PD-1 blockade exposure (FIG. 4C). Overall, these data indicate that intravesical delivery of EcN^{hCXCL13} promotes immunotherapy responsiveness in a model of PD-1 resistant bladder tumors.

The antitumor activity of this combination regimen in the MB49 orthotopic model was subsequently validated by starting the treatment 10 days after bladder implantation of the MB49 cells to generate advanced bladder tumors (FIG. 3C). Intravesical delivery of EcN^{hCXCL13} once a week for 4 weeks was combined with 4 injections of PD-1 blockade every 3-4 days after treatment initiation. EcN^{hCXCL13} and PD-1 blockade significantly extended survival, with ˜47% (n=7/15) of mice surviving and remaining tumor-free after >150 days of follow-up compared to ˜27% (n=3/11) of mice surviving and tumor-free after treatment with EcN⁻ or PBS combined with PD-1 blockade (FIG. 3D). Notably, this correlated with a significant increase in median survival of 105days after EcN^{hCXCL13} plus PD-1 blockade combination therapy, as compared to 34 and 57 days after EcN⁻ or PBS plus PD-1 blockade, respectively.

To evaluate the robustness and durability of this effect, the surviving mice were re-challenged by subcutaneously injecting MB49 cells on dual flanks as a model of distant metastasis. In contrast to age-matched naïve controls (n=6/6) and surviving mice treated with anti-PD-1 plus EcN⁻ (n=1/3) or PBS (n=1/3), tumor engraftment was not observed in any (n=0/7) of the surviving mice that had previously been treated with EcN^{hCXCL13} and PD-1 blockade (FIG. 3E). Together, these observations highlight the potent antitumor activity of EcN^{hCXCL13} combined with PD-1 blockade, which significantly enhanced long-term survival and provided durable protection against metastatic rechallenge in an aggressive orthotopic model of bladder cancer.

### Example 17

### T Follicular Helper Cells Drive the Therapeutic Efficacy of EcN^{hCXCL13} Plus PD-1 Blockade

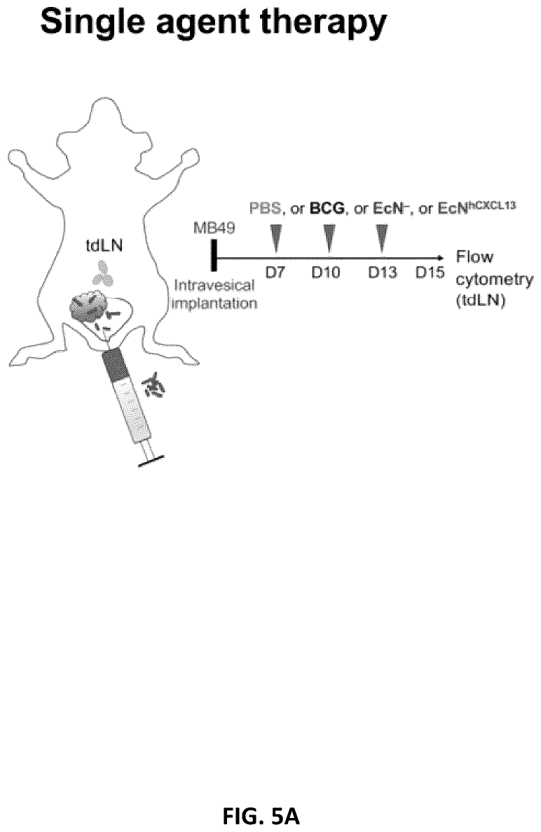

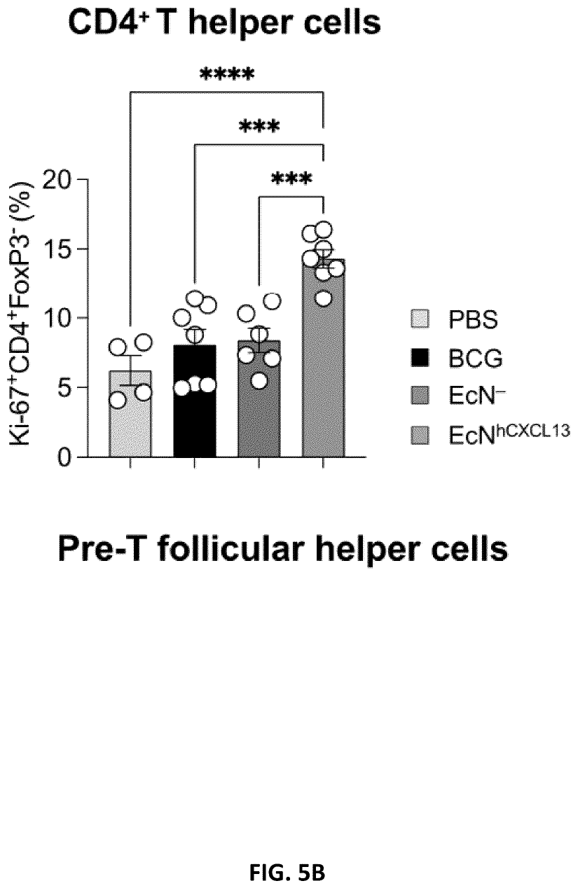

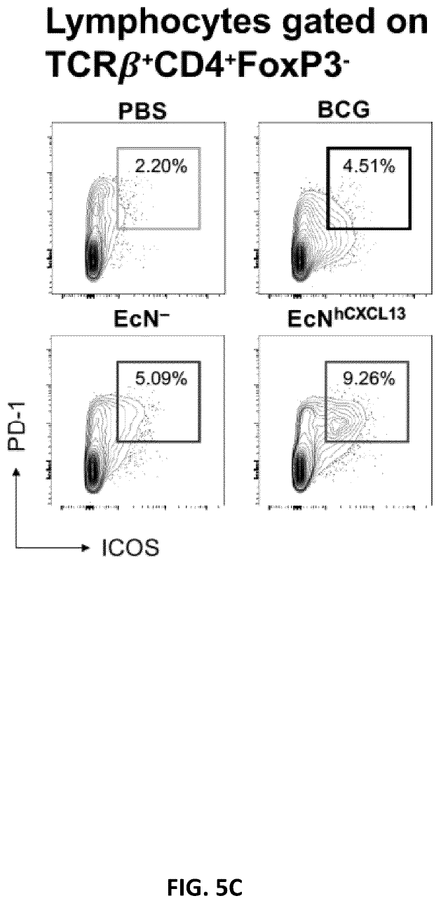

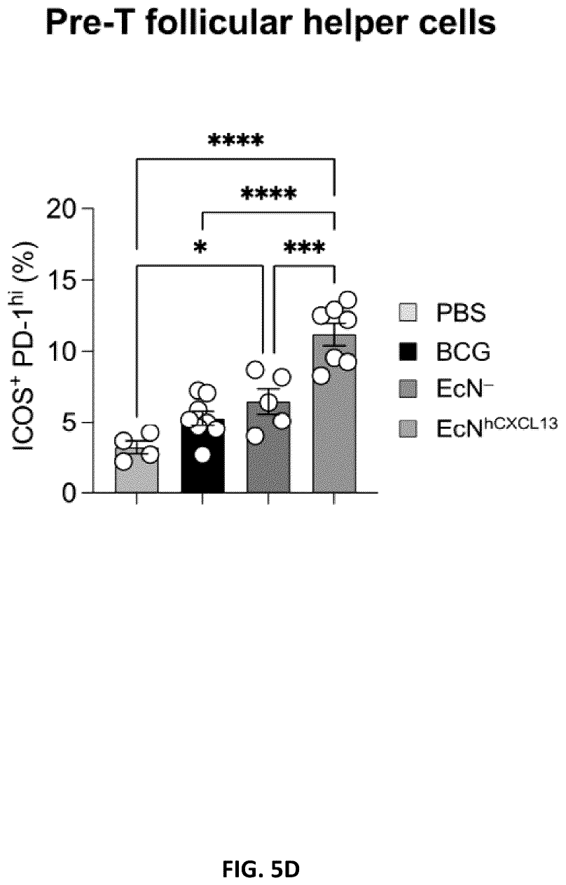

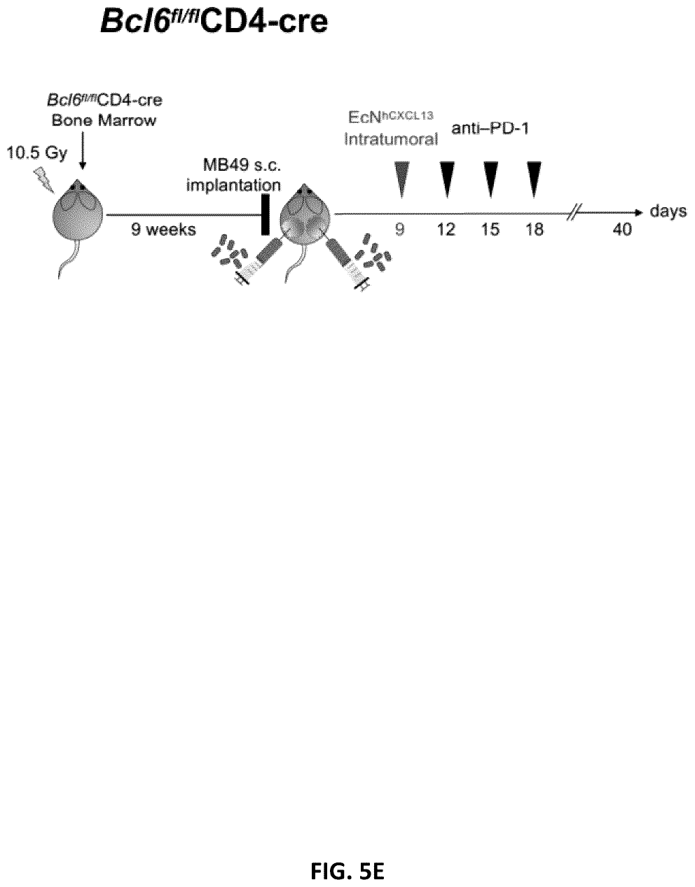

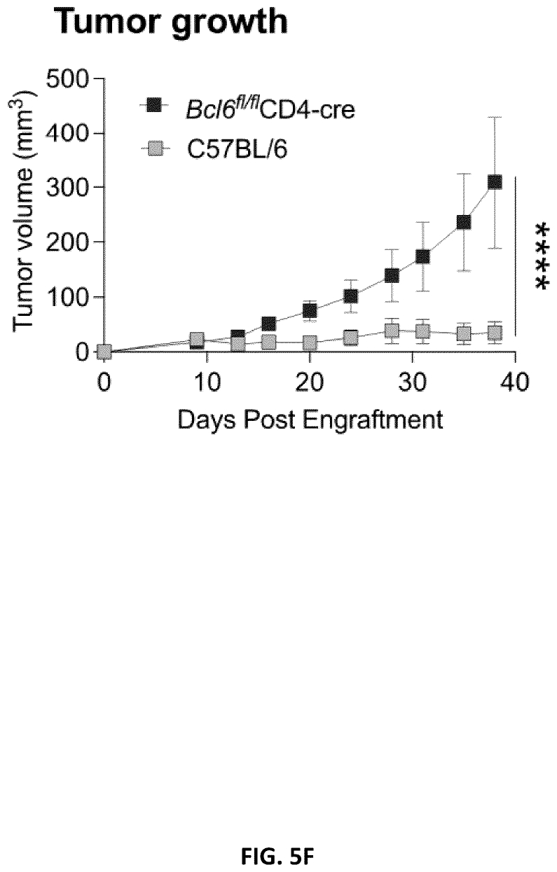

To gain insights into the mechanisms by which EcN^{hCXCL13} promotes responsiveness to PD-1 blockade, the expression of key molecules associated with the follicular program and function of CD4⁺ Tfh cells was examined. To this end, the proliferation of CD4⁺ T helper cells was evaluated in bladder tumor-draining lymph nodes in mice receiving intravesical administrations of EcN^{hCXCL13}, EcN⁻, PBS, or bacillus Calmette-Guerin (BCG), a live attenuated vaccine approved for bladder cancer therapy which is known to induce CD4⁺ T cell-dependent tumor-specific immunity in an MB49 orthotopic model of bladder cancer (FIG. 5A). A significant increase in the frequencies of Ki-67⁺CD4⁺FoxP3⁻T cells was observed after intravesical delivery of EcN^{hCXCL13} as compared with EcN⁻, BCG, or PBS, suggesting that EcN^{hCXCL13} is sufficient to activate naïve CD4⁺helper T cells in bladder tumor draining lymph nodes (FIG. 5B).

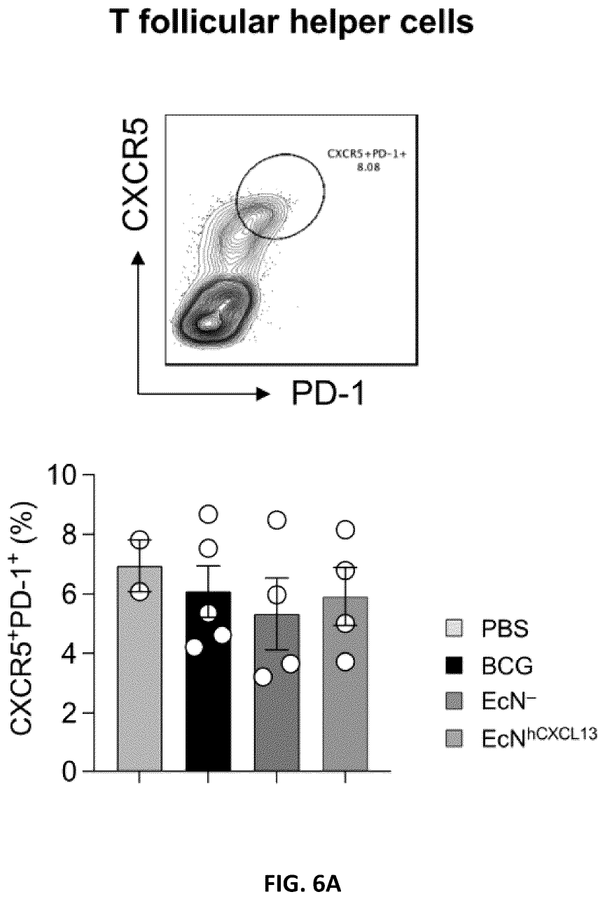

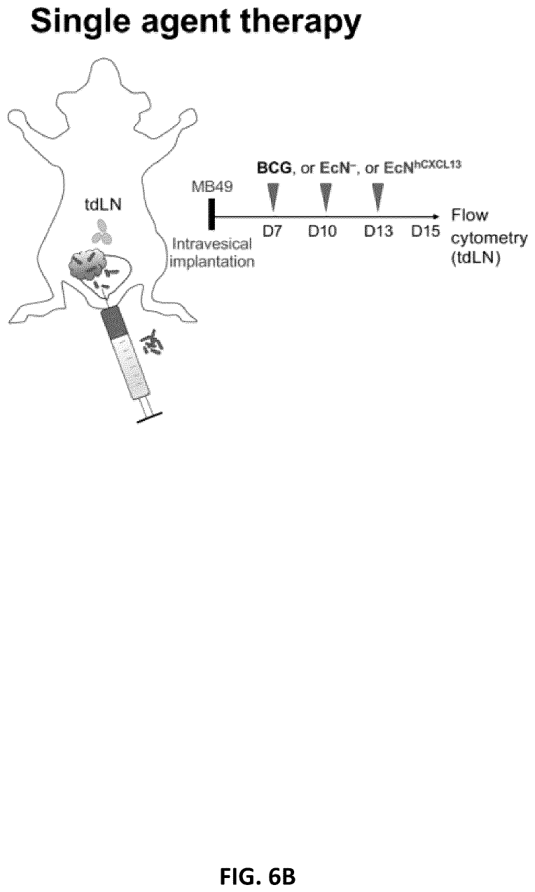

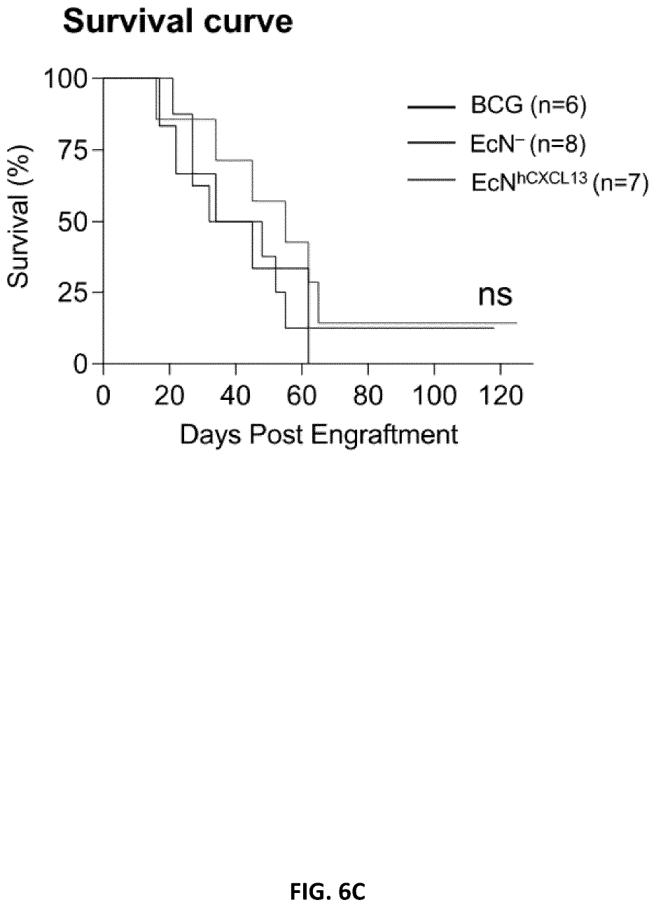

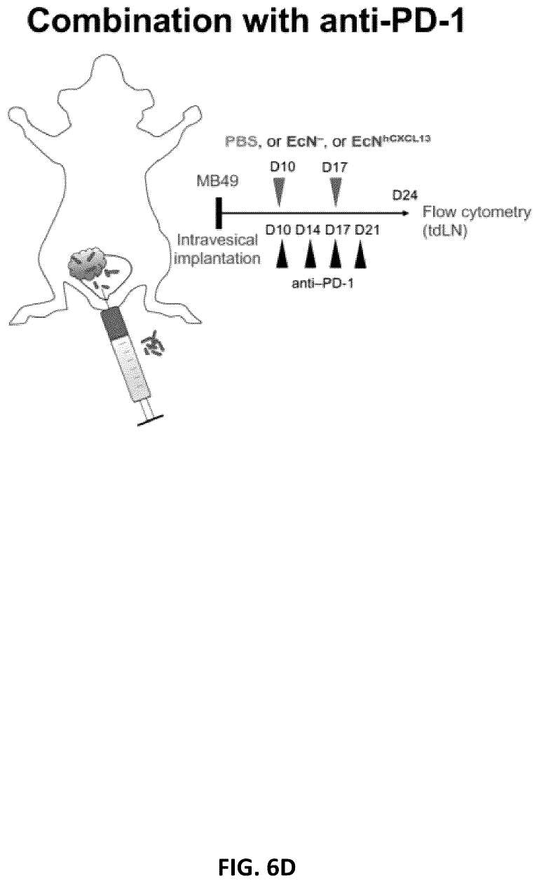

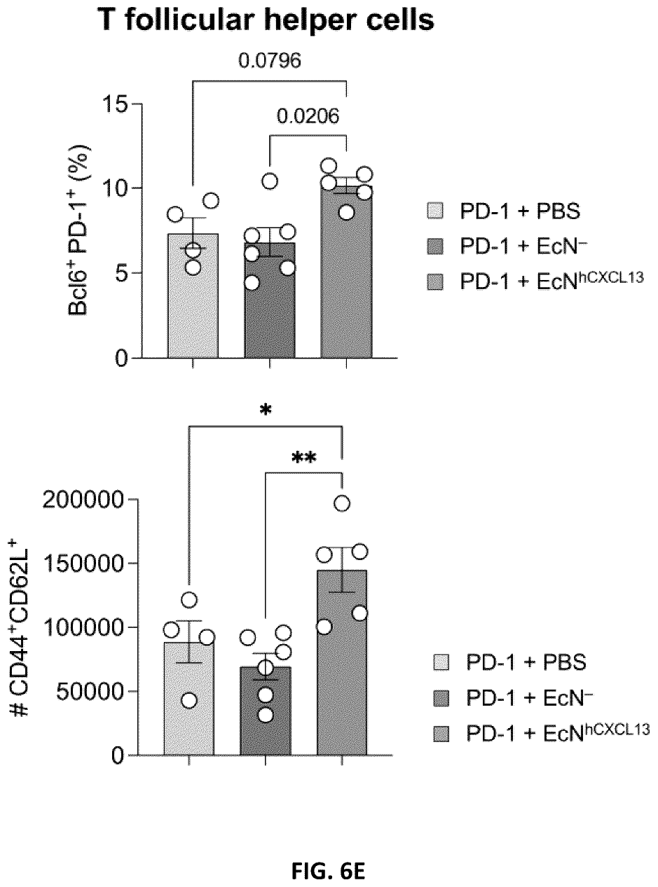

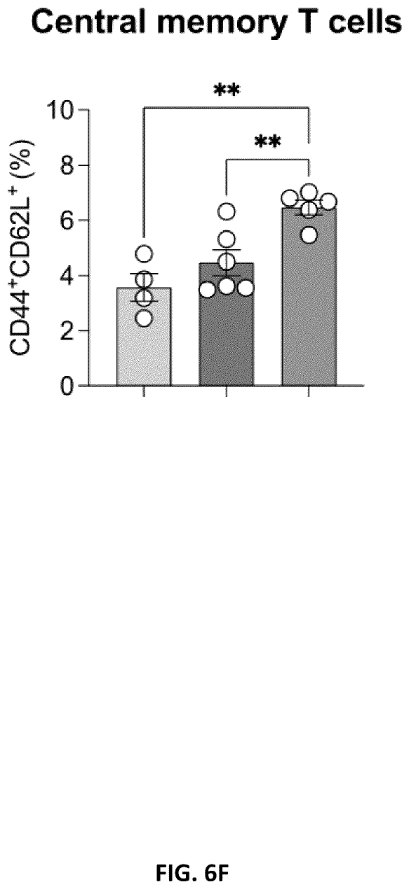

The co-stimulatory molecule ICOS and the inhibitory receptor PD-1 play a central role in establishing the Tfh program and controlling the homing and positioning of CD4⁺T cells in the B cell follicle during the GC response. Analysis revealed that ICOS and PD-1 co-expression significantly increased in CD4⁺ helper T cells after intravesical delivery of EcN^{hCXCL13} as compared with EcN⁻, BCG, or PBS (FIG. 5C and 5D). However, we did not observe any upregulation of CXCR5, similar to previous observations in Tfh cells from human cancer-associated tissues (FIG. 6A). Of note, the median survival of mice treated with EcN^{hCXCL13} alone was 53 days, as compared with 39 days and 37 days in mice treated with EcN⁻ or BCG, respectively (FIG. 6B and 6C). These observations confirm that EcN^{hCXCL13} fosters the activation of CD4⁺T helper cells with a pre-Tfh phenotype but has limited therapeutic effect when used as a single therapeutic agent.

To confirm that activated CD4⁺ cells expressing high levels of PD-1 molecules may respond to PD-1 blockade, the frequency of Tfh cells co-expressing PD-1 and the Tfh-defining transcription factor Bcl6 was measured following intravesical delivery of EcN^{hCXCL13} combined with PD-1 blockade. In this setting, a significant increase in the frequencies of bladder tumor-draining lymph node Tfh cells (Bcl6⁺PD-1⁺) was observed upon treatment with EcN^{hCXCL13} plus PD-1 blockade as compared to mice treated with EcN⁻ or PBS plus PD-1 blockade, suggesting a dynamic effect of PD-1 blockade on Tfh differentiation (FIG. 6D and 6E). Analyses also revealed a significant increase in the frequencies and total numbers of CD4⁺CD62L⁺CD44⁺ bladder tumor-draining lymph node central memory T cells in mice treated with combination EcN^{hCXCL13} plus PD-1 blockade therapy, which further highlights a potential role of memory T cells induced by our engineered probiotic therapy in the dynamics of the GC reaction (FIG. 6F).

To further investigate the role of Tfh cells in driving the therapeutic efficacy of EcN^{hCXCL13} plus PD-1 blockade, animals deficient in Tfh production were generated by transplanting bone marrow from Bel6^{fl/fl}CD4-cre mice into lethally irradiated C57BL/6 recipient animals. Following reconstitution, MB49 cells were subcutaneously implanted into the hind flanks of Tfh-deficient bone marrow chimeras or age-matched wild-type control animals. At 9 days post-engraftment, mice received a single intratumoral injection of EcN^{hCXCL13}, followed by intraperitoneal injections of anti-PD-1 antibody (FIG. 5E). As in the orthotopic setting, treatment with EcN^{hCXCL13} plus PD-1 blockade mediated potent antitumor control in wild-type; however, in bone marrow chimeric mice lacking Tfh cells (Bcl6^{fl/fl}CD4-cre), the therapeutic benefit of EcN^{hCXCL13}plus PD-1 blockade combination therapy was abolished (FIG. 5F). Altogether, these findings demonstrate that EcN^{hCXCL13} plus PD-1 blockade induces the differentiation of CD4⁺T helper cells into Tfh cells, which ultimately mediate the antitumor efficacy afforded by this combination therapy.

### Example 18

### EcN^{hCXCL13} Plus PD-1 Blockade Boost the Production of Antitumor Antibodies

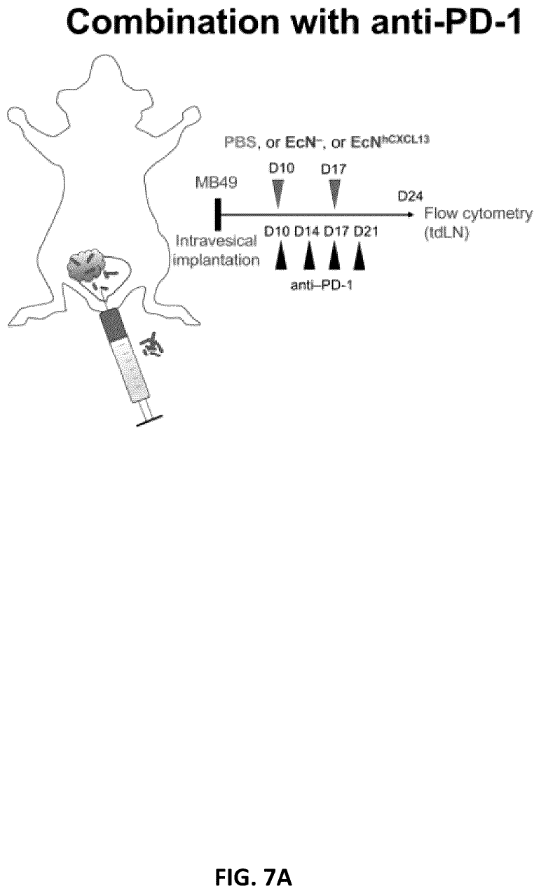

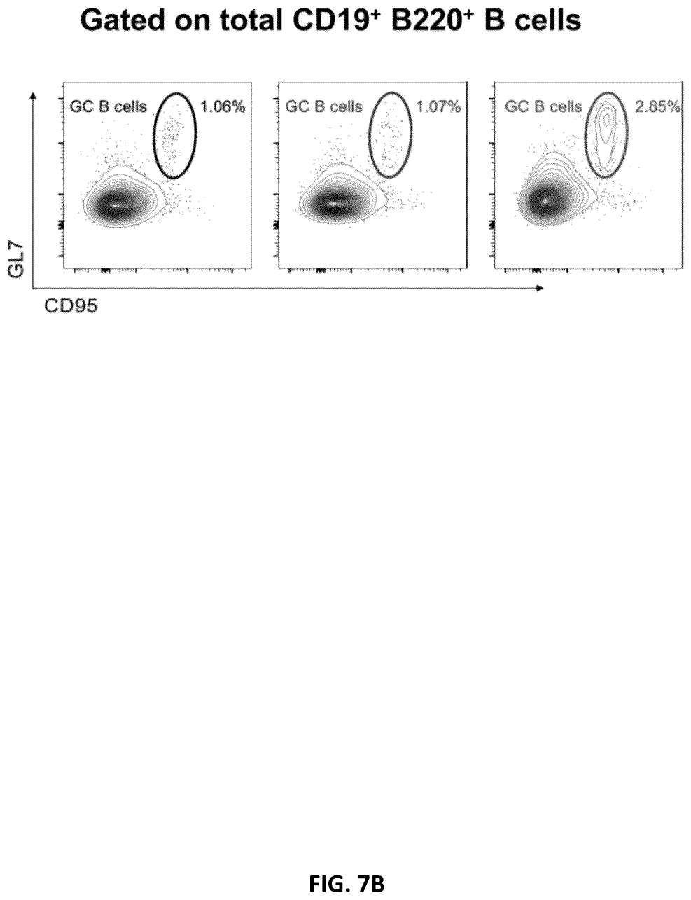

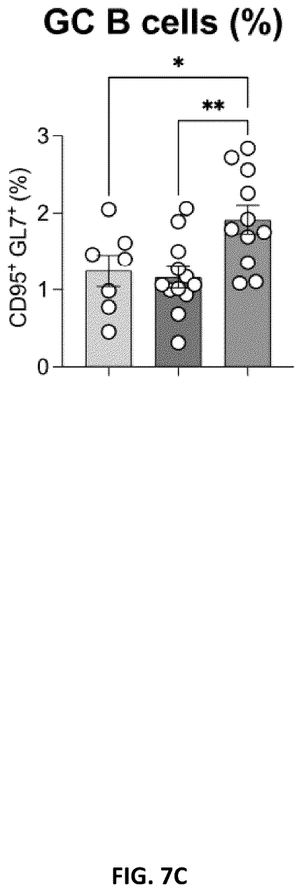

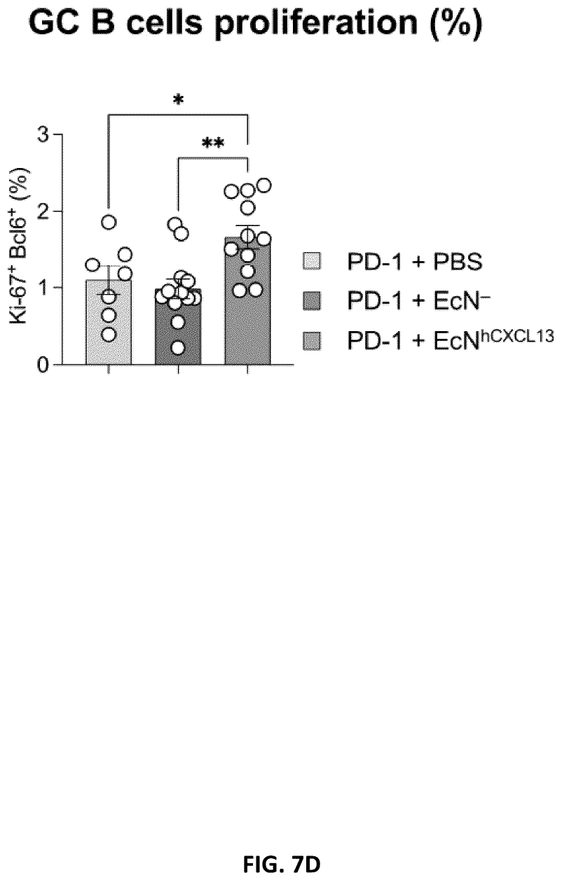

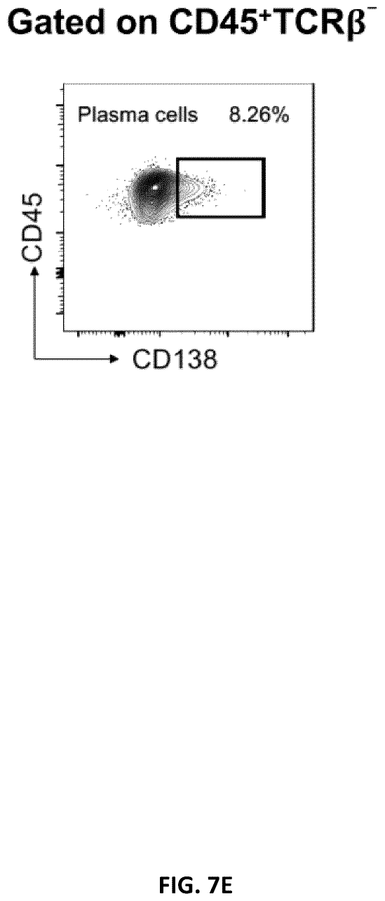

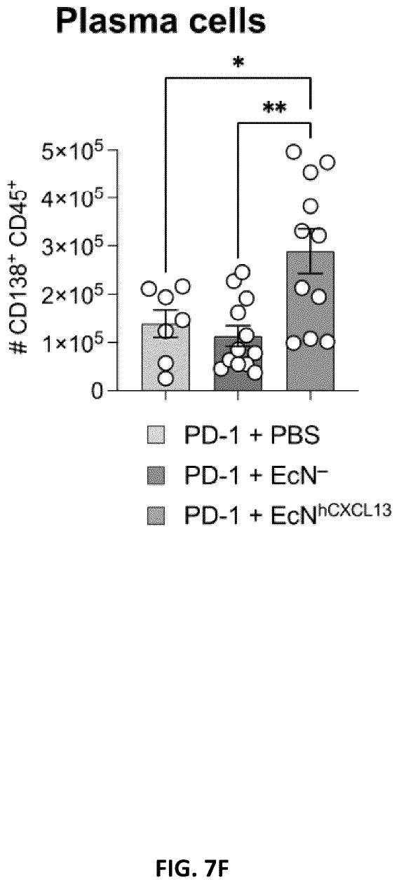

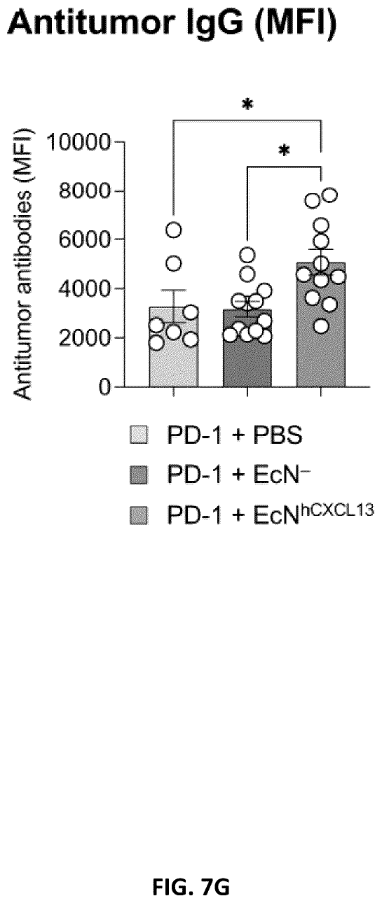

The immune phenotype of GC B cells was assessed by flow cytometry following treatment of MB49 bladder tumor-bearing mice with intravesical EcN^{hCXCL13}, EcN⁻, or PBS, plus PD-1 blockade (FIG. 7A). As compared to mice receiving EcN⁻ or PBS plus PD-1 blockade, upon treatment with EcN^{hCXCL13} plus PD-1 blockade, a marked increase in the frequencies of B220⁺CD19⁺GL7⁺CD95⁺ GC B cells was observed, along with increased frequencies of proliferating GC B cells (Ki-67⁺Bcl6⁺) (FIG. 7B-7D). These data demonstrate that EcN^{hCXCL13} plus PD-1 blockade enhances the GC reaction in bladder tumor-draining lymph nodes.

To evaluate GC B cell differentiation into antibody-secreting cells, the frequency of CD138⁺ plasma cells in bladder tumor draining lymph nodes was assessed. Analysis of CD45+ immune cells by flow cytometry showed a ˜3-fold increase of plasma cells upon EcN^{hCXCL13} plus PD-1 blockade therapy, as compared to EcN⁻ or PBS plus PD-1 blockade (FIG. 7E and 7F). These data suggest that actively proliferating GC B cells expand in response to EcN^{hCXCL13} plus PD-1 blockade and further differentiate into plasma cells.

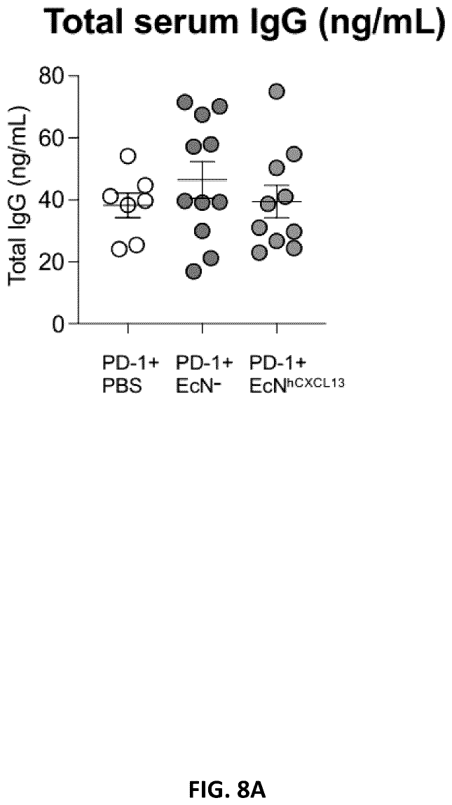

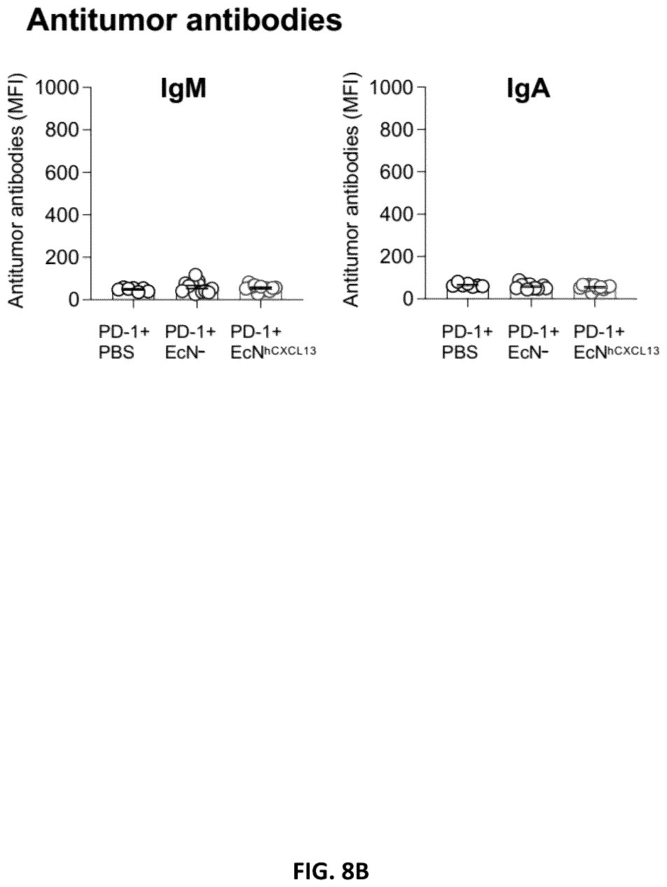

To investigate the potential production of tumor-specific antibodies by plasma cells, endpoint sera from each treatment group of MB49 bladder tumor-bearing mice were incubated in vitro with cultured MB49 cells. Whereas the total level of circulating IgG antibodies was similar in each treatment group, as compared to mice treated with EcN⁻ or PBS plus PD-1 blockade, a significant increase in the specificity and/or binding affinity of serum antibodies for MB49 cells was observed for sera isolated from mice treated with EcN^{hCXCL13} plus PD-1 blockade (FIG. 7G, FIG. 8A). Circulating IgM and IgA antibodies did not demonstrate any detectable binding to MB49 bladder cancer cells (FIG. 8B). Together, these observations demonstrate that EcN^{hCXCL13} plus PD-1 blockade promotes the GC reaction and plasma cell differentiation, resulting in the production of tumor-specific IgG antibodies in an immunocompetent model of bladder cancer.

While this invention has been disclosed with reference to particular embodiments, it is apparent that other embodiments and variations of the inventions disclosed herein can be devised by others skilled in the art without departing from the true spirit and scope thereof. The appended claims include all such embodiments and equivalent variations.

What is claimed is:

1. A method of treating muscle-invasive bladder cancer in a subject comprising administering a programmable bacterial cell to the subject, wherein the programmable bacterial cell comprises:
(a) a synchronized lysis circuit comprising a first nucleic acid encoding a quorum-sensing gene, a second nucleic acid encoding a lysis gene, a promoter, and a terminator contained on a single operon; and
(b) a third nucleic acid encoding CXCL13.

2. The method of claim 1, wherein the programmable bacterial cell further comprises a fourth nucleic acid encoding a therapeutic agent selected from the group consisting of an anti-PD-1 antibody and an anti-PD-L1 antibody.

3. The method of claim 1, wherein the programmable bacterial cell belongs to a genus selected from the group consisting of Salmonella, Escherichia, Firmicutes, Bacteroidetes, Lactobacillus, and Bifidobacteria.

4. The method of claim 3, wherein the programmable bacterial cell belongs to the genus Escherichia.

5. The method of claim 4, wherein the programmable bacterial cell is an Escherichia coli Nissle 1917 (EcN) cell.

6. The method of claim 1, wherein a plurality of the programmable bacterial cells are administered to the subject at least four times.

7. The method of claim 6, wherein the plurality of the programmable bacterial cells are administered to the subject once a week for at least four weeks.

8. A programmable bacterial cell comprising:
(a) a synchronized lysis circuit comprising a first nucleic acid encoding a quorum-sensing gene, a second nucleic acid encoding a lysis gene, a promoter, and a terminator contained on a single operon; and
(b) a third nucleic acid encoding CXCL13.

9. The programmable bacterial cell of claim 8, further comprising a fourth nucleic acid encoding a therapeutic agent selected from the group consisting of an anti-PD-1 antibody and an anti-PD-L1 antibody.

10. The programmable bacterial cell of claim 8, wherein the programmable bacterial cell belongs to a genus selected from the group consisting of Salmonella, Escherichia, Firmicutes, Bacteroidetes, Lactobacillus, and Bifidobacteria.

11. The programmable bacterial cell of claim 10, wherein the programmable bacterial cell belongs to the genus Escherichia.

12. The programmable bacterial cell of claim 11, wherein the programmable bacterial cell is an Escherichia coli Nissle 1917 (EcN) cell.
